(19) United States
(12) Patent Application Publication
(10) Pub. No.: US 2007/0099831 A1
(43) Pub. Date: May 3, 2007

(54) Parathyroid hormone analogues and methods of use

(76) Inventor: Paul Morley, Ottawa (CA)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/517,146

(22) Filed: Sep. 6, 2006

Related U.S. Application Data
(60) Provisional application No. 60/714,905, filed on Sep. 6, 2005.
(60) Provisional application No. 60/834,980, filed on Jul. 31, 2006.
(60) Provisional application No. 60/837,972, filed on Aug. 15, 2006.

(51) Int. Cl. A61K 38/29; A61K 31/59; A61K 33/06

(52) U.S. Cl. 514/12; 514/167; 424/682

(57) ABSTRACT

The present invention is directed to novel methods of treating a subject with a bone deficit disorder. The methods generally include administering to a subject in need thereof a pharmaceutically acceptable formulation comprising a parathyroid hormone (PTH) peptide analogue in a daily dose of 2 μg to 60 μg, wherein said PTH peptide analogue has a reduced phospholipase-C activity and maintains adenylate cyclase activity.

### RELATED APPLICATIONS

This application claims benefit of priority to U.S. Provisional Application No. 60/714,905, filed Sep. 6, 2005, and U.S. Provisional Application No. 60/834,980, filed Jul. 31, 2006, and U.S. Provisional Application No. 60/837,972, filed Aug. 15, 2006, the entire contents of each of which are incorporated herein by reference.

### BACKGROUND OF THE INVENTION

Bone remodeling, or turnover, consists of two opposing activities: the breakdown (resorption) of old bone by osteoclasts, and the formation of new bone by osteoblasts. Loss of bone mass occurs as part of the natural aging process. Calcium is constantly being added to and taken away from bone. When calcium is taken away faster than it is added, the bones become lighter, less dense, and more porous. This makes the bones weaker and increases their risk of fracture.

Bones naturally become thinner (called osteopenia) as people grow older, because existing bone is broken down faster than new bone is made. As this occurs, the bones lose minerals, heaviness (mass), and structure, making them weaker and more fragile. With further bone loss, osteopenia develops into osteoporosis. Accordingly, the thicker a person's bones are, the longer it takes to develop osteoporosis. Although osteoporosis can occur in men, it is most common in women older than age 65.

Osteoporosis often results in spontaneous fractures of load-bearing bones and the physical and mental deterioration characteristic of immobilizing injuries. In particular, postmenopausal osteoporosis is caused by the disappearance of estrogens which triggers an acceleration of bone turnover with an increased imbalance between resorption of old bone and formation of new bone. Instead of bone mass remaining stable, bone loss results because osteoclasts, the cells that destroy old bone (resorption of bones), outperform osteoblasts, the cells that build new bone (formation of bones). This accelerated bone loss due to resorption without adequate compensation by bone formation results in gradual thinning, increased porosity, and depletion of load-bearing bones.

End stage renal disease is invariably associated with bone disease, known as renal osteodystrophy (ROD). ROD may exist in a high turnover form characterized by high circulating levels of parathyroid hormone (PTH) and overactive bone tissue, often with osteitis fibrosa cystica. The low turnover form of the disease, also known as adynamic bone disease, is characterized by normal or low circulating levels of PTH. Histologically, the bone surfaces are quiescent with little or no cellular activity and osteomalacia may also be present. The incidence of the condition is increased with advanced age, presence of corticosteroid therapy, presence of calcimimetic therapy, calcium containing phosphate binders and high doses of Vitamin D sterols. However, adynamic bone disease is currently difficult to treat without leading to an unacceptable increase in serum calcium. Accordingly, there is a continuous unmet need for effective therapy.

Among the remedies for osteoporosis (which have historically involved increase in dietary calcium, estrogen therapy, and increased doses of vitamin D), human parathyroid hormone (hPTH) treatments are used to build bones to compensate for the bone loss due to osteoporosis. Parathyroid hormone is produced by the parathyroid gland and is involved in the control of calcium levels in blood. It is a hypercalcemic hormone, elevating blood calcium levels. PTH is a polypeptide and synthetic polypeptides may be prepared using the method disclosed by Erickson and Merrifield, The Proteins, Neurath et al., Eds., Academic Press, New York, 1976, page 257, preferably as modified by the method of Hodges et al., Peptide Research, 1, 19 (1988) or by Atherton, E. and Sheppard, R. C., Solid Phase Peptide Synthesis, IRL Press, Oxford, 1989. When serum calcium is reduced to below a “normal” level, the parathyroid gland releases PTH and resorption of bone calcium and increased absorption of calcium from the intestine, as well as renal reabsorption of calcium, occur. An antagonist of PTH is calcitonin, which acts to reduce the level of circulating calcium. Although high levels of PTH can remove calcium from the bone, intermittent low doses can actually promote bone growth. For example, the native hPTH-(1-84) and its fragment hPTH-(1-34) (as sold under the tradename FORTEO® by Eli Lilly and Co.) have been shown to be useful in the treatment of osteoporosis. The native hPTH-(1-84) and the hPTH-(1-34) fragment, however, suffer a drawback that while they promote bone formation, they simultaneously activate bone resorption. As a consequence hPTH-(1-34) is effective in reducing the fracture frequency of trabecular bone (which make up the bones of the axial skeleton, and include the rib cage, the back bones and the skull, and vertebrate bone), but its fracture reduction efficacy on cortical bone (which serves to protect against torsional loads and includes, for example, the hip and wrists) is considerably less.

There remains a need for therapeutic approaches employing suitable PTH analogues to restore bones and increase bone mineral density in both trabecular and cortical bones in patients with osteoporosis or other bone degenerative/deficit disorders. There further remains a need for therapeutic approaches employing suitable PTH analogues to restore bones and increase bone mineral density and formation without stimulating bone resorption, and without significantly increasing the levels of serum calcium in patients with osteoporosis or other bone degenerative disorders.

### SUMMARY OF THE INVENTION

The present invention provides pharmaceutical compositions and formulations containing suitable PTH peptide analogues for use in methods directed to treating subjects suffering from various bone degenerative or bone deficit disorders. The PTH peptide analogue compounds described herein induce bone formation in both trabecular and cortical bones, thereby increasing bone mineral density and restoring bones. Unexpectedly, the PTH peptide analogues described herein induce bone formation while causing less bone resorption than previously known PTH analogues, and also demonstrate lower incidences of and severity in hypercalcemia.

The PTH analogues disclosed herein, when administered within the specified dosage ranges, are effective in reversing the effects of osteoporosis on cortical bones in animals. Righting the imbalance between resorption of old cortical bone and formation of new cortical bone, these PTH analogues have been shown to reverse the effects of osteoporosis on bone. Thus, the methods described herein promote cortical bone growth in animals without significantly increasing cortical bone porosity.

These PTH analogues also promote recovery from bone injuries. Therefore, administration of the specified dosages of the PTH analogues of the present invention restore osteoporotic cortical bones and promote bone healing in various circumstances, such as in the treatment of fractures.

In one aspect, the invention provides a method for the treatment of osteoporosis, comprising administering to a subject in need thereof a pharmaceutically acceptable formulation comprising a parathyroid hormone (PTH) peptide analogue in a daily dose of 2 μg to 60 μg, wherein said PTH peptide analogue has a reduced phospholipase-C activity and maintains adenylate cyclase activity.

In another embodiment, the invention is directed to a method for treating a bone fracture, comprising administering to a subject in need thereof a pharmaceutically acceptable formulation comprising a parathyroid hormone (PTH) peptide analogue in a daily dose of 2 μg to 60 μg, wherein said PTH peptide analogue has reduced phospholipase-C activity and maintains adenylate cyclase activity.

In another embodiment, the invention provides a method of inducing bone formation in trabecular and cortical bones, comprising administering to a subject in need thereof a pharmaceutically acceptable formulation comprising a parathyroid hormone (PTH) peptide analogue in a daily dose of 2 μg to 60 μg, wherein said PTH peptide analogue has reduced phospholipase-C activity and maintains adenylate cyclase activity.

In yet another embodiment, the present invention is directed to a method of treating or preventing renal osteodystrophy (ROD) and related disorders, comprising administering to a subject in need thereof a pharmaceutically acceptable formulation comprising a parathyroid hormone (PTH) peptide analogue in a daily dose of 2 μg to 60 μg, wherein said PTH peptide analogue has reduced phospholipase-C activity and maintains adenylate cyclase activity.

Another embodiment provides the use of the PTH peptides of the present invention for treating osteoporosis, for treating or preventing a bone fracture, for inducing bone formation in trabecular and cortical bones, for treating or preventing renal osteodystrophy (ROD) and related disorders, or for any other therapeutic use of PTH wherein calcium monitoring is not required.

Another embodiment provides the use of the PTH peptides of the present invention for treating osteoporosis, for treating or preventing a bone fracture, for inducing bone formation in trabecular and cortical bones, for treating or preventing renal osteodystrophy (ROD) and related disorders, or for any other therapeutic use of PTH, wherein a warning regarding osteosarcoma formation is not required.

In another embodiment, the invention provides a pharmaceutical formulation comprising a unit dosage form of a therapeutically effective amount of a parathyroid hormone (PTH) peptide analogue in a daily dosage range of 2 to 60 μg, wherein said PTH peptide analogue has reduced phospholipase-C activity and maintains adenylate cyclase activity; and a pharmaceutically acceptable excipient, diluent, or carrier, or combinations thereof.

Another embodiment of the invention is a kit for treating a bone deficit disorder comprising, in one or more containers, a therapeutically effective amount of the above-described pharmaceutical composition contained in a device, and a label or packaging insert containing instructions for use.

PTH analogues optionally include less than the first 34 amino acids at the N-terminal end. The PTH peptide analogues of the present invention, when compared to full-length PTH peptides or other PTH peptide analogues which are 34 amino acid residues or longer, trigger less than full activation of phospholipase-C, less bone resorption, and less incidences or lower severity of hypercalcemia, while still maintaining increases in bone mineral density (BMD) at a variety of sites within the body.

Specific embodiments of PTH peptide analogues of the present invention include the following: PTH-(1-31)NH2, Ostabolin; PTH-(1-30)NH2; PTH-(1-29)NH2; PTH-(1-28)NH2; Leu27PTH-(1-31)NH2; Leu27PTH-(1-30)NH2; Leu27PTH-(1-29)NH2; Leu27cyclo(22-26)PTH-(1-31)NH2 Ostabolin-C™; Leu27cyclo(22-26)PTH-(1-34)NH2; Leu27cyclo(Lys26-Asp30)PTH-(1-34)NH2; Cyclo(Lys27-Asp30)PTH-(1-34)NH2; Leu27cyclo(22-26)PTH-(1-31)NH2; Ala27 or Nle27 or Tyr27 or Ile27 cyclo(22-26)PTH-(1-31)NH2; Leu27cyclo(22-26)PTH-(1-32)NH2; Leu27cyclo(22-26)PTH-(1-31)OH; Leu27cyclo(26-30)PTH-(1-31)NH2; Cys22Cys26Leu27cyclo(22-26)PTH-(1-31)NH2; Cys22Cys26Leu27cyclo(26-30)PTH-(1-31)NH2; Cyclo(27-30)PTH-(1-31)NH2; Leu27cyclo(22-26)PTH-(1-30)NH2; Cyclo(22-26)PTH-(1-31)NH2; Cyclo(22-26)PTH-(1-30)NH2; Leu27cyclo(22-26)PTH-(1-29)NH2; Leu27cyclo(22-26)PTH-(1-28)NH2; Glu17,Leu27cyclo(13-17)(22-26)PTH-(1-28)NH2; and Glu17,Leu27cyclo(13-17)(22-26)PTH-(1-31)NH2.

The PTH peptides of the present invention can be administered at a variety of doses, most preferably at a daily dose of 5, 10, 15, 20, 25, or 30 μg.

### BRIEF DESCRIPTION OF THE DRAWINGS

In the figures described below, if not stated otherwise, the measurements following administration of Ostabolin-C™ were made after a 15 week course of subcutaneous daily administration of the stated dose, and the changes were measured as compared to baseline. As used herein, baseline is the patient's individual measurement prior to receiving any treatment.

FIG. 1 is a bar graph showing the percentage change in lumbar spine bone mineral density (BMD) in patients with moderate osteoporosis receiving a pharmaceutical formulation containing [Leu²⁷]cyclo[Glu²²-Lys²⁶]-PTH-(1-31)-NH₂.

FIG. 2 is a graph showing the percentage change in lumbar spine bone mineral density (BMD) in patients with moderate osteoporosis receiving the pharmaceutical formulation containing hPTH-(1-34) teriparatide, Forteo®.

FIG. 3 is a bar graph showing the percentage change in total hip bone mineral density (BMD) in patients with moderate osteoporosis receiving a pharmaceutical formulation containing [Leu²⁷]cyclo[Glu²²-Lys²⁶]-PTH-(1-31)-NH₂.

FIG. 4 is a bar graph showing the percentage change in femoral neck bone mineral density (BMD) in patients with moderate osteoporosis receiving a pharmaceutical formulation containing [Leu²⁷]cyclo[Glu²²-Lys²⁶]-PTH-(1-31)-NH₂.

FIG. 5 is a bar graph showing the percentage change in trochanter bone mineral density (BMD) in patients with moderate osteoporosis receiving a pharmaceutical formulation containing [Leu²⁷]cyclo[Glu²²-Lys²⁶]-PTH-(1-31)-NH₂.

FIG. 6 is a bar graph showing the percentage change in distal radius bone mineral density (BMD) in patients with moderate osteoporosis receiving a pharmaceutical formulation containing [Leu²⁷]cyclo[Glu²²-Lys²⁶]-PTH-(1-31)-NH₂.

FIG. 7 is a bar graph showing the percentage change in mid-shaft radius bone mineral density (BMD) in patients with moderate osteoporosis receiving a pharmaceutical formulation containing [Leu²⁷]cyclo[Glu²²-Lys²⁶]-PTH-(1-31)-NH₂.

FIG. 8 is a bar graph showing the percentage change in the bone formation marker amino terminal pro-peptide of type I pro-collagen (P1INP) in patients with moderate osteoporosis receiving a pharmaceutical formulation containing [Leu²⁷]cyclo[Glu²²-Lys²⁶]-PTH-(1-31)-NH₂.

FIG. 9 is a bar graph showing the percentage change in the bone formation marker osteocalcin in patients with moderate osteoporosis receiving a pharmaceutical formulation containing [Leu²⁷]cyclo[Glu²²-Lys²⁶]-PTH-(1-31)-NH₂.

FIG. 10 is a bar graph showing the percentage change in the bone formation marker bone-specific alkaline phosphatase (BSAP) in patients with moderate osteoporosis receiving a pharmaceutical formulation containing [Leu²⁷]cyclo[Glu²²-Lys²⁶]-PTH-(1-31)-NH₂.

FIG. 11 is a bar graph showing the percentage change in the bone resorption marker N-telopeptide (NTx) in patients with moderate osteoporosis receiving a pharmaceutical formulation containing [Leu²⁷]cyclo[Glu²²-Lys²⁶]-PTH-(1-31)-NH₂.

FIG. 12 is a bar graph showing the percentage change in the bone resorption marker C-terminal telopeptide (CTx) in patients with moderate osteoporosis receiving a pharmaceutical formulation containing [Leu²⁷]cyclo[Glu²²-Lys²⁶]-PTH-(1-31)-NH₂.

FIG. 13 is a graph showing the percentage change in the bone formation and bone resorption markers in patients with moderate osteoporosis receiving the pharmaceutical formulation containing rhPTH-(1-34), teriparatide, Forteo®.

FIG. 14 is a bar graph showing the percentage of abnormal serum calcium levels in patients with moderate osteoporosis receiving a pharmaceutical formulation containing [Leu²⁷]cyclo[Glu²²-Lys²⁶]-PTH-(1-31)-NH₂.

FIG. 15 is a slide showing the Forteo data derived from Deal et al., (2005) J. Bone Min. Res. 20, p. 1905-1991.

FIG. 16 is a slide showing the effectiveness of Ostabolin-C and Forteo.

FIG. 17 is a slide showing the effectiveness of Ostabolin-C and Forteo.

### DETAILED DESCRIPTION OF THE INVENTION

The present invention provides pharmaceutical compositions and formulations containing suitable PTH peptide analogues for use in methods directed to treating subjects suffering from various bone degenerative or bone deficit disorders. The PTH peptide analogue compounds described herein induce bone formation in both trabecular and cortical bones, thereby increasing bone mineral density and restoring bones. Unexpectedly, the PTH peptide analogues described herein induce bone formation while causing less bone resorption than previously known PTH analogues, and also demonstrate lower incidences and severity of hypercalcemia.

The invention relates to a method for increasing bone toughness and/or stiffness, and/or reducing incidence of fracture in a subject by administering a parathyroid hormone. The method can be employed to increase stiffness and/or toughness at a site of a potential trauma or at a site of an actual trauma. Trauma generally includes fracture, surgical trauma, joint replacement, orthopedic procedures, and the like. Increasing bone toughness and/or stiffness generally includes increasing mineral density of cortical bone, increasing strength of bone, increasing resistance to loading, and the like. Reducing incidence of fracture generally includes reducing the likelihood or actual incidence of fracture for a subject compared to an untreated control population.

The present invention includes a method for increasing the toughness and/or stiffness of bone, including trabecular and cortical bone, and/or reducing the incidence and/or severity of fracture by administering a parathyroid hormone analogue as described herein. More particularly, the invention relates to a method for increasing toughness or stiffness of bone at a site of a potential or actual trauma. Increasing toughness and/or stiffness of bone can be manifested in numerous ways known to those of skill in the art, such as increasing bone mineral density, increasing bone mineral content, increasing work to failure, and the like. In one embodiment, the method of the invention reduces the incidence or severity of vertebral and/or non-vertebral fractures. The method of the invention can be used to decrease the risk of such fractures or for treating such fractures. In particular, the method of the invention can reduce the incidence of vertebral and/or non-vertebral fracture, reduce the severity of vertebral fracture, reduce the incidence of multiple vertebral fracture, improve bone quality, and the like.

The inventors have discovered that PTH peptide analogues that have a reduced phospholipase-C activity, and which maintain adenylate cyclase activity, are surprisingly useful for inducing bone formation in both trabecular and cortical bones, and causing less bone resorption than previous PTH analogues at dosages of about 2 to about 60 μg/day, without significantly increasing levels of serum calcium. The methods provided by this invention are generally practiced by administering to an animal in need thereof a dose of a PTH compound in the amount of about 2 to about 60 μg/day, to induce bone formation and cause less bone resorption and lower incidences of hypercalcemia as compared to the administration of PTH analogues 34 amino acid residues in length or longer.

The PTH peptide analogues, either alone or in combination with other bone enhancing agents, of the present invention can be used to treat any mammal, including humans and animals, suffering from a disease, symptom, or condition related to bone deficiency. In an embodiment of the invention, the subject in need of enhanced bone formation is a human patient such as a man or a woman. In a preferred embodiment, the patient is a post-menopausal woman.

Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the “PTH peptide analogues” of the present invention are preferably, but not exclusively, non-naturally occurring and may be obtained either recombinantly or by peptide synthesis. The PTH analogues of the present invention include fragments or variants of fragments of human, rat, porcine, or bovine PTH that have human PTH activity as determined in the ovarectomized rat model of osteoporosis. Kimmel et al., Endocrinology, 1993, 32(4):1577. Human PTH activity includes the ability of the PTH to increase trabecular and/or cortical bone growth. The PTH analogues of the present invention increase AC activity when administered to a PTH receptor containing cell in culture, such as an osteoclast. The PTH analogues of the present invention have certain additional functional activities, as defined below.

As used herein, a PTH peptide analogue that has a “reduced phospholipase-C activity” refers to a PTH peptide analogue that has been truncated or modified in some manner so as to trigger less than full activation of phospholipase-C, as compared to the full-length PTH peptide or other PTH peptide analogues which are at least 34 amino acid residues in length.

As used herein, a PTH peptide analogue that leads to “reduced bone resorption” refers to a PTH peptide analogue that has been truncated or modified in some manner so as to trigger less bone resorption, as compared to the full-length PTH peptide or other PTH peptide analogues which are at least 34 amino acid residues in length.

As used herein, a PTH peptide analogue that leads to “reduced hypercalcemia levels” refers to a PTH peptide analogue that has been truncated or modified in some manner so as to trigger less incidences of hypercalcemia, or lower severity of hypercalcemia, as compared to the full-length PTH peptide or other PTH peptide analogues which are at least 34 amino acid residues in length.

As used herein, “treating” or “treatment of” a condition or subject refers to taking steps to obtain beneficial or desired results, including clinical results. For purposes of this invention, beneficial or desired clinical results include, but are not limited to, alleviation or amelioration of one or more disease, symptom, or condition related to bone deficiency. Generally, such bone deficit disease, symptoms, and conditions are treated by inducing bone formation as measured by an increase in bone mineral density (“BMD”). For example, symptoms of osteoporosis include back pain, loss of height and stooped posture, a curved backbone (dowager's hump), or fractures that may occur with a minor injury (especially of the hip, spine, or wrist). Symptoms of Paget's disease most commonly include bone pain. Other symptoms can include: headaches and hearing loss, neck pain, pressure on nerves, increased head size or bending of spine, hip pain, damage to cartilage of joints (which may lead to arthritis), and Barrel-shaped chest. Symptoms of osteoarthritis can include joint pain and aching, limited range of motion and instability, radiographic evidence of the erosion of the articular cartilage, joint space narrowing, sclerosis of the subchondral bone, and osteophytes (spurs). Symptoms for rheumatoid arthritis include painful, swollen, tender, stiff joints on both sides of the body (symmetrical), especially the hands, wrists, elbows, feet, knees, or neck. Rheumatoid nodules (bumps) ranging in size from a pea to a mothball develop in nearly one-third of people who have rheumatoid arthritis. These nodules usually form over pressure points in the body such as the elbows, knuckles, spine, and lower leg bones.

As used herein, “reduction” of a symptom or symptoms (and grammatical equivalents of this phrase) means decreasing of the severity or frequency of the symptom(s), or elimination of the symptom(s).

As used herein, “administering” or “administration of” a drug or pharmaceutical composition or formulation described herein to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

A variety of administration routes can be used in accordance with the present invention. An effective amount of the peptide described herein can be administered parenterally, orally, by inhalation, topically, rectally, nasally, buccally, vaginally or via an implanted reservoir.

In a preferred embodiment of the invention, an effective amount of the peptide described herein can be administered parenterally. The term “parenteral” as used herein includes subcutaneous, intravenous, intramuscular, intra-articular, intra-synovial, intrastemal, intrathecal, intrahepatic, intralesional and intracranial injection or infusion techniques. More preferably, the route of administration is subcutaneous administration.

As used herein, a “therapeutically effective amount” of a drug or pharmaceutical composition or formulation, or agent, described herein is an amount of a drug or agent that, when administered to a subject with a disease or condition, will have the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

As used herein, a “prophylactically effective amount” of a drug or pharmaceutical composition or formulation, or agent, described herein is an amount of a drug or agent that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a prophylactically effective amount may be administered in one or more administrations.

Administration of a bone enhancing agent “in combination with” a drug or pharmaceutical composition or formulation described herein includes parallel administration (i.e., administration of both the drug and the agents to the subject over a period-of time, co-administration (in which both the drug and agents are administered at approximately the same time, e.g., within about a few minutes to a few hours of one another), and co-formulation (in which both the drug and agents are combined or compounded into a single dosage form suitable for oral or parenteral administration).

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

Bone Disorders and Diseases

Bone Deficits

In one aspect, the subject in need has a bone deficit, which means that they will have less bone than desirable or that the bone will be less dense or strong than desired. A bone deficit may be localized, such as that caused by a bone fracture or systemic, such as that caused by osteoporosis. Bone deficits may result from a bone remodelling disorder whereby the balance between bone formation and bone resorption is shifted, resulting in a bone deficit. Examples of such bone remodelling disorders include, for example, osteoporosis, Paget's disease, renal osteodystrophy, renal rickets, osteoarthritis, rheumatoid arthritis, achondroplasia, osteochodrytis, hyperparathyroidism, osteogenesis imperfecta, congenital hypophosphatasia, fribromatous lesions, fibrous displasia, multiple myeloma, abnormal bone turnover, osteolytic bone disease and periodontal disease. Bone remodelling disorders includes metabolic bone diseases which are characterized by disturbances in the organic matrix, bone mineralization, bone remodelling, endocrine, nutritional and other factors which regulate skeletal and mineral homeostasis. Such disorders may be hereditary or acquired and generally are systemic, affecting the entire skeletal system.

Thus, in one aspect the human subject may have a bone remodelling disorder. Bone remodelling as used herein refers to the process whereby old bone is being removed and new bone is being formed by a continuous turnover of bone matrix and mineral that involves bone resorption by osteoclasts and bone formation by osteoblasts.

Osteoporosis is a common bone remodelling disorder characterised by a decrease in bone density of normally mineralised bone, resulting in thinning and increased porosity of bone cortices and trabeculae. The skeletal fragility caused by osteoporosis predisposes sufferers to bone pain and an increased incidence of fractures. Progressive bone loss in this condition may result in a loss of up to 50% of the initial skeletal mass. Primary osteoporosis includes idiopathic osteoporosis which occurs in children or young adults with normal gonadal function, Type I osteoporosis, also described as post-menopausal osteoporosis, and Type II osteoporosis, senile osteoporosis, occurs mainly in those persons older than 70 years of age. Causes of secondary osteoporosis may be endocrine (e.g., glucocorticoid excess, hyperparathyroidism, hypoganodism), drug induced (e.g. corticosteroid, heparin, tobacco) and miscellaneous (e.g., chronic renal failure, hepatic disease and malabsorbtion syndrome osteoporosis).

The phrase “at risk of developing a bone deficit”, as used herein, is intended to embrace subjects having a higher than average predisposition towards developing a bone deficit. As an example, those susceptible towards osteoporosis include post-menopausal women, elderly males (e.g., those over the age of 65) and those being treated with drugs known to cause osteoporosis as a side-effect (e.g., steroid-induced osteoporosis). Certain factors are well known in the art which may be used to identify those at risk of developing a bone deficit due to bone remodelling disorders like osteoporosis. Risk factors for osteoporosis are known in the art and include hypogonadal conditions in men and women, irrespective of age, conditions, diseases or drugs that induce hypogonadism, nutritional factors associated with osteoporosis (low calcium or vitamin D being the most common), smoking, alcohol, drugs associated with bone loss (such as glucocorticoids, thyroxine, heparin, lithium, anticonvulsants etc.), loss of eyesight that predisposes to falls, space travel, immobilization, chronic hospitalization or bed rest, and other systemic diseases that have been linked to increased risk of osteoporosis.

Indications of the presence of osteoporosis are known in the art and include radiological evidence of at least one vertebral compression fracture, low bone mass (typically at least 1 standard deviation below mean young normal values), and/or atraumatic fractures. Other important factors include family history, life style, estrogen or androgen deficiency and negative calcium balance. Postmenopausal women are particularly at risk of developing osteoporosis. Hereinafter, references to treatment of bone diseases are intended to include management and/or prophylaxis except where the context demands otherwise.

Bone Trauma

The method of the invention is of benefit to a subject that may suffer or have suffered trauma to one or more bones. The method can benefit mammalian subjects, such as humans, horses, dogs, and cats, in particular, humans. Bone trauma can be a problem for racing horses and dogs, and also for household pets. A human can suffer any of a variety of bone traumas due, for example, to accident, medical intervention, disease, or disorder. In the young, bone trauma is likely due to fracture, medical intervention to repair a fracture, or the repair of joints or connective tissue damaged, for example, through athletics. Other types of bone trauma, such as those from osteoporosis, degenerative bone disease (such as arthritis or osteoarthritis), hip replacement, or secondary conditions associated with therapy for other systemic conditions (e.g., glucocorticoid osteoporosis, burns or organ transplantation) are found most often in older people.

Osteoporosis can lead, for example, to vertebral and/or non-vertebral fractures. Vertebral fractures are those involving the spinal column and non-vertebral fractures refers to any fracture not involving the spinal column. Non-vertebral fractures are more common than fractures of the vertebrae—an estimated 850,000 non-vertebral compared with 700,000 vertebral fractures occur annually in the United States. Non-vertebral fractures include more than 300,000 hip and 250,000 wrist fractures, in addition to 300,000 fractures at other non-vertebral sites. Other examples of non-vertebral fractures include a hip fracture, a fracture of a distal forearm, a fracture of a proximal humerus, a fracture of a wrist, a fracture of a radius, a fracture of an ankle, a fracture of an humerus, a fracture of a rib, a fracture of a foot, a fracture of a pelvis, or a combination of these.

The method of the invention can be used to decrease the risk of such fractures or for treating such fractures. The risk of fracture is diminished and the healing of a fracture is aided by increasing the strength and/or stiffness of bone, for example, in the hip, the spine or both. A typical woman at risk for osteoporosis is a postmenopausal woman or a premenopausal, hypogonadal woman. A preferred subject is a postmenopausal woman, and is independent of concurrent hormone replacement therapy (HRT), estrogen or equivalent therapy, or antiresorptive therapy. The method of invention can benefit a subject at any stage of osteoporosis, but especially in the early and advanced stages.

The present invention provides a method, in particular, effective to prevent or reduce the incidence of fractures in a subject with or at risk of progressing to osteoporosis. For example, the present invention can reduce the incidence of vertebral and/or non-vertebral fracture, reduce the severity of vertebral fracture, reduce the incidence of multiple vertebral fracture, improve bone quality, and the like. In another embodiment, the method of the present invention can benefit patients with low bone mass or prior fracture who are at risk for future multiple skeletal fractures, such as patients in which spinal osteoporosis may be progressing rapidly.

Other subjects can also be at risk of or suffer bone trauma and can benefit from the method of the invention. For example, a wide variety of subjects at risk of one or more of the fractures identified above, can anticipate surgery resulting in bone trauma, or may undergo an orthopedic procedure that manipulates a bone at a skeletal site of abnormally low bone mass or poor bone structure, or deficient in mineral. For example, recovery of function after a surgery such as a joint replacement (e.g. knee or hip) or spine bracing, or other procedures that immobilize a bone or skeleton can improve due to the method of the invention. The method of the invention can also aid recovery from orthopedic procedures that manipulate a bone at a site of abnormally low bone mass or poor bone structure, which procedures include surgical division of bone, including osteotomies, joint replacement where loss of bone structure requires restructuring with acetabulum shelf creation and prevention of prosthesis drift, for example. Other suitable subjects for practice of the present invention include those suffering from hypoparathyroidism or kyphosis, who can undergo trauma related to, or caused by, hypoparathyroidism or progression of kyphosis.

Bone Toughness and Stiffness

The method of the invention reduces the risk of trauma or aids recovery from trauma by increasing bone toughness, stiffness or both. Generally toughness or stiffness of bone results from mass and strength of cortical and trabecular (cancellous) bone. The method of the invention can provide levels of bone toughness, stiffness, mass, and/or strength within or above the range of the normal population. Preferably the invention provides increased levels relative to the levels resulting from trauma or giving rise to risk of trauma. Increasing toughness, stiffness, or both decreases risk or probability of fracture compared to an untreated control population.

Certain characteristics of bone when increased provide increased bone toughness and/or stiffness. Such characteristics include bone mineral density (BMD), bone mineral content (BMC), activation frequency or bone formation rate, trabecular number, trabecular thickness, trabecular and other connectivity, periosteal and endocortical bone formation, cortical porosity, cross sectional bone area and bone mass, resistance to loading, and/or work to failure. An increase in one or more of these characteristics is a preferred outcome of the method of the invention.

Certain characteristics of bone, such as marrow space and elastic modulus when decreased provide increased toughness and/or stiffness of bone. Younger (tougher and stiffer) bone has crystallites that are generally smaller than crystallites of older bone. Thus, gerierally reducing the size of bone crystallites increases toughness and stiffness of bone, and can reduce incidence of fracture. In addition, maturing the crystallites of a bone can provide additional desirable characteristics to the bone, including increased toughness and stiffness of bone and/or can reduced incidence of fracture. A decrease in one or more of these characteristics can be a preferred outcome of the method of the invention.

The method of the invention is effective for increasing the toughness and/or stiffness of any of several bones. For example, the present method can increase the toughness and/or stiffness of bones including a hip bone, such as an ilium, a leg bone, such as a femur, a bone from the spine, such as a vertebra, or a bone from an arm, such as a distal forearm bone or a proximal humerus. This increase in toughness and/or stiffness can be found throughout the bone, or localized to certain portions of the bone. For example, toughness and/or stiffness of a femur can be increased by increasing the toughness and/or stiffness of a femur neck or a femur trochanter. Toughness and/or stiffness of a hip can be increased by increasing the toughness and/or stiffness of an iliac crest or iliac spine. Toughness and/or stiffness of a vertebra can be increased by increasing the toughness and/or stiffness of a pedicle, lamina, or body. Advantageously, the effect is on vertebra in certain portions of the spine, such as cervical, thoracic, lumbar, sacral, and/or coccygeal vertebrae. Preferably the effect is on one or more mid-thoracic and/or upper lumbar vertebrae.

The increase in toughness and/or stiffness can be found in each of the types of bone, or predominantly in one type of the bone. Types of bone include spongy (cancellous, trabecular, or lamellar) bone and compact (cortical or dense) bone and the fracture callus. The method of the invention preferably increases toughness and/or stiffness through its effects on cancellous and cortical bone, or on cortical bone alone. Trabecular bone, bone to which connective tissue is attached can also be toughened and/or stiffened by the present method. For example, it is advantageous to provide additional toughness at a site of attachment for a ligament, a tendon, and/or a muscle.

In another aspect of the invention, increasing toughness or stiffness can reduce incidence of fracture. In this aspect, increasing toughness or stiffness can include reducing incidence of vertebral fracture, reducing incidence of severe fracture, reducing incidence of moderate fracture, reducing incidence of non-vertebral fracture, reducing incidence of multiple fracture, or a combination thereof.

The methods of the invention may also be used to enhance bone formation in conditions where a bone deficit is caused by factors other than bone remodelling disorders. Such bone deficits include fractures, bone trauma, conditions associated with post-traumatic bone surgery (e.g., bone grafts or bone fusions), post-prosthetic joint surgery, post plastic bone surgery, post dental surgery, bone chemotherapy, and bone radiotherapy. Fractures include all types of microscopic and macroscopic fractures. Examples of fractures and/or injuries include avulsion fracture, comminuted fracture, non-union fracture, transverse fracture, oblique fracture, spiral fracture, segmental fracture, a segmental gap, displaced fracture, impacted fracture, greenstick fracture, torus fracture, fatigue fracture, intra-articular fracture (epiphyseal fracture), closed fracture (simple fracture), open fracture (compound fracture), a bone void, and occult fracture in any bones of the subject.

As previously mentioned, a wide variety of bone diseases may be treated in accordance with the present invention, for example all those bone diseases connected with the bone-remodelling cycle. Examples of such diseases include all forms of osteoporosis, osteomalacia and rickets. Osteoporosis, especially of the post-menopausal, male, post-transplant, and steroid-induced types, is of particular note. In addition, PTH peptide analogues find use as bone promotion agents, and as anabolic bone agents. Such uses form another aspect of the present invention.

Parathyroid Hormone Analogues

As active ingredient, the pharmaceutically acceptable composition or solution described herein may incorporate fragments, or variants of fragments, including substitutions, deletions, or insertions, of human PTH, or of rat, porcine or bovine PTH that have human PTH activity as determined in the ovarectomized rat model of osteoporosis reported by Kimmel et al., Endocrinology, 1993, 32(4):1577. Human PTH activity includes the ability of the PTH to increase trabecular and/or cortical bone growth. The PTH analogues of the present invention increase AC activity when administered to a PTH receptor containing cell in culture, such as an osteoclast. The PTH analogues used in the present invention are naturally or non-naturally occurring and desirably incorporate less than the first 34 N-terminal residues of PTH.

PTH operates through activation of two second messenger systems, Gₛ-protein activated adenylyl cyclase (AC) and G_{q}-protein activated phospholipase C. The latter results in a stimulation of membrane-bound protein kinase Cs (PKC) activity. The PKC activity has been shown to require PTH residues 29 to 32 (Jouishomme et al (1994) J. Bone Mineral Res. 9, (1179-1189). It has been established that the increase in bone growth, i.e. that effect which is useful in the treatment of osteoporosis, is coupled to the ability of the peptide sequence to increase AC activity.

The native PTH sequence, and its truncated 1-34 form, has been shown to have all of these activities. The hPTH-(1-34) sequence is:

(SEQ ID NO:1)

Ser Val Ser Glu Ile Gln Leu Met His Asn Leu Gly
Lys His Leu Asn Ser Met Glu Arg Val Glu Trp Leu
Arg Lys Lys Leu Gln Asp Val His Asn Phe-OH

AC activity has been shown to require the first few N-terminal residues of the molecule. Thus, in accordance with this embodiment of the invention, it is possible to remove those biological activities associated with the PKC activity by deleting a selected terminal portion of the hPTH-(1-34) molecule. In one embodiment, these shortened analogues are desirably in the form of carboxyl terminal amides. One feature of the invention therefore comprises variants of the human parathyroid analogues PTH(1-25)-NH₂, PTH(1-26)-NH₂, PTH(₁-27)-NH₂, PTH(1-28)-NH₂, PTH(1-29)-NH₂, PTH(1-30)-NH₂, and PTH(1-31)-NH₂.

According to another feature of the PTH analogues to be used in the present invention, it has surprisingly been found that replacing Lys₂₇ with a Leu in the native hPTH sequence results in a higher activity for AC stimulation. This analogue also exhibits its maximum activity when in the form of the carboxyl terminal amide. Thus, another feature of the invention comprises the use of PTH analogues including all sequences from [Leu₂₇]-PTH-(1-25)-NH₂ to [Leu₂₇]-PTH-(1-31)-NH₂.

According to another feature of the present invention, lactams of the PTH analogues are formed, for example, by cyclisation involving the coupling of the side-chains of Glu22 and Lys26, or of the side-chains Lys26 and Asp30, in which Lys27 may be replaced by a Leu or by various other hydrophobic residues, and which has either a C-terminal free amide ending, or has a C-terminal free carboxyl ending. Such substitutions include ornithine, citrulline, alpha-aminobutyric acid, or any linear or branched alpha-amino aliphatic acid, having 2-10 carbons in the side chain, any such analogue having a polar or charged group at the terminus of the aliphatic chain. Example of polar or charged groups include amino, carboxyl, acetamido, guanido and ureido. Ile, norleucine, Met, and ornithine are expected to be the most active.

The PTH analogues of the present invention may thus feature the formation of a lactam, for example, between either residues Glu22 and Lys26, Ly26 and Asp30, or Glu22 and Lys27. The substitution of Leu for the Lys27 results in a more hydrophobic residue on the hydrophobic face of the amphiphilic helix. This resulted in increased adenylyl cyclase stimulating activity in the PTH receptor containing rat osteosarcoma (ROS) cell line. It will be appreciated by those skilled in the art that other such substitutions would likely result in analogues with the same or increased activities. These hydrophobic substitutions include residues such as Met or norleucine. The combined effect of substitution and either lactam formation is expected to stabilize the alpha-helix and increase bioactivity, and to protect this region of the molecule from proteolytic degradation. The presence of the amide at the C-terminus is further expected to protect the peptide against exoproteolytic degradation (Leslie, F. M. and Goldstein, A. (1982) Neuropeptides 2, 185-196).

In one preferred embodiment of the invention, the peptide used in the disclosed method is PTH(1-31)-NH2 with the following sequence:

(SEQ ID NO:2)

Ser-Val-Ser-Glu-Ile-Gln-Leu-Met-His-Asn-Leu-Gly-
Lys-His-Leu-Asn-Ser-Met-Glu-Arg-Val-Glu-Trp-Leu-
Arg-Lys-Xaa-Leu-Gln-Asp-Val-NH₂.

Xaa is selected from the group consisting of Lys, Leu, Ile, Nle and Met. In a preferred embodiment, Xaa is Lys (SEQ ID NO: 3). This embodiment is also referred to as OSTABOLIN.

In another preferred embodiment of the invention, the peptide used in the disclosed method is cyclo(22-26)PTH-(1-31)-NH2, cyclized in the form of a lactam between Glu²² and Lys²⁶ with the following sequence:

(SEQ ID NO:4)

Ser-Val-Ser-Glu-Ile-Gln-Leu-Met-His-Asn-Leu-Gly-
Lys-His-Leu-Asn-Ser-Met-Glu-Arg-Val-Glu-Trp-Leu-
Arg-Lys-Xaa-Leu-Gln-Asp-Val-Y,

Xaa is selected from the group consisting of Leu, Ile, Nle and Met and Y is NH₂ or OH. When Xaa is Leu and Y is NH₂ (SEQ ID NO: 5), the PTH is also referred to as OSTABOLIN-C™.

The PTH analogues to be used in the present invention can thus be cyclized or linear, and can be optionally amidated at the C-terminus. Alternatives in the form of PTH variants incorporate from 1 to 5 amino acid substitutions that improve PTH stability and half-life, such as the replacement of methionine residues at positions 8 and/or 18 with leucine or other hydrophobic amino acid that improves PTH stability against oxidation and the replacement of amino acids in the 25-27 region with trypsin-insensitive amino acids such as histidine or other amino acid that improves PTH stability against protease. Other suitable forms of PTH include PTHrP, PTHrP(1-34), PTHrP(1-36) and analogs of PTH or PTHrP that activate the PTH1 receptor. These forms of PTH are embraced by the term “parathyroid hormone analogues” as used generically herein. The hormones may be obtained by known recombinant or synthetic methods, such as described in U.S. Pat. Nos. 4,086,196; 5,556,940; 5,955,425; 6,541,450; 6,316,410; and 6,110,892, incorporated herein by reference.

Specific embodiments of PTH peptide analogues of the present invention include the following: PTH-(1-31)NH2, Ostabolin; PTH-(1-30)NH2; PTH-(1-29)NH2; PTH-(1-28)NH2; Leu27PTH-(1-31)NH2; Leu27PTH-(1-30)NH2; Leu27PTH-(1-29)NH2; Leu27cyclo(22-26)PTH-(1-31)NH2 Ostabolin-C™; Leu27cyclo(22-26)PTH-(1-34)NH2; Leu27cyclo(Lys26-Asp30)PTH-(1-34)NH2; Cyclo(Lys27-Asp30)PTH-(1-34)NH2; Leu27cyclo(22-26)PTH-(1-31)NH2; Ala27 or Nle27 or Tyr27 or Ile27 cyclo(22-26)PTH-(1-31)NH2; Leu27cyclo(22-26)PTH-(1-32)NH2; Leu27cyclo(22-26)PTH-(1-31)OH; Leu27cyclo(26-30)PTH-(1-31)NH2; Cys22Cys26Leu27cyclo(22-26)PTH-(1-31)NH2; Cys22Cys26Leu27cyclo(26-30)PTH-(1-31)NH2; Cyclo(27-30)PTH-(1-31)NH2; Leu27cyclo(22-26)PTH-(1-30)NH2; Cyclo(22-26)PTH-(1-31)NH2; Cyclo(22-26)PTH-(1-30)NH2; Leu27cyclo(22-26)PTH-(1-29)NH2; Leu27cyclo(22-26)PTH-(1-28)NH2; Glu17,Leu27cyclo(13-17)(22-26)PTH-(1-28)NH2; and Glu17,Leu27cyclo(13-17)(22-26)PTH-(1-31)NH2.

Generally, preferred embodiments of PTH peptide analogues include those that when administered result in reduced phospholipase-C activity, reduced bone resorption, and reduced hypercalcemia levels. As defined in the Definitions section herein, “reduced phospholipase-C activity” refers to a PTH peptide analogue that has been truncated or modified in some manner so as to trigger less than full activation of phospholipase-C, as compared to the full-length PTH peptide or other PTH peptide analogues which are at least 34 amino acid residues in length; “reduced bone resorption” refers to a PTH peptide analogue that has been truncated or modified in some manner so as to trigger less bone resorption, as compared to the full-length PTH peptide or other PTH peptide analogues which are at least 34 amino acid residues in length, and “reduced hypercalcemia levels” refers to a PTH peptide analogue that has been truncated or modified in some manner so as to trigger less incidences of hypercalcemia, or lower severity of hypercalcemia, as compared to the full-length PTH peptide or other PTH peptide analogues which are at least 34 amino acid residues in length.

The preferred PTH analogues administered in the methods described herein include [Leu²⁷]cyclo[Glu²²-Lys26]-PTH-(1-31)-NH₂, such as advanced by Zelos Therapeutics, Inc. under the tradename OSTABOLIN-C™ and [Leu²⁷] PTH-(1-31)-NH². In another embodiment of the invention, [Leu²⁷]cyclo[Glu²²-Lys²⁶]-PTH-(1-30)-NH₂ is used in the methods described herein. In another embodiment, the hormone can be the linear analogue PTH(1-31), which can have a free carboxyl ending, or be amidated, at the C-terminus. In yet another embodiment, the hormone can be PTH(1-30), which can have a free carboxyl ending, or be amidated, at the C-terminus; or [Leu2]-PTH(1-30)-NH₂. Suitable stabilized solutions of these and other PTH analogues that can be employed in the present methods are described in U.S. Pat. Nos. 5,556,940; 5,955,425; 6,541,450; 6,316, 410; and 6,110,892 incorporated herein by reference.

Methods of the Invention and Agents Useful Therein

The methods provided by this invention are generally practiced by administering to an animal in need thereof a daily or weekly dose of a PTH compound in an amount effective to induce bone formation and inhibit or reduce bone loss or resorption.

One aspect of the present invention provides a method for treating osteoporosis by administering to a subject in need thereof a pharmaceutically acceptable formulation comprising a PTH peptide analogue in a daily dose of 2 μg to 60 μg or a weekly dose of from 14 μg to 420 μg , wherein the PTH peptide analogue has a reduced phospholipase-C activity but maintains adenylate cyclase activity. In one embodiment, the subject is a human man or woman. In a preferred embodiment the woman is post-menopausal.

In another embodiment, the osteoporosis can be selected from the group consisting of advanced-stage osteoporosis, hypogonadal osteoporosis, spinal osteoporosis, transplant-induced osteoporosis, and steroid-induced osteoporosis.

Bone enhancing agents known in the art to increase bone formation, bone density or bone mineralisation, or to prevent bone resorption may be used in the methods and pharmaceutical compositions of the invention. Those of ordinary skill in the bone formation art also recognize that suitable bone enhancing agents include, for example, natural or synthetic hormones, such as selective estrogen receptor modulators (SERMs), estrogens, androgens, calcitonin, prostaglandins and parathormone; growth factors, such as platelet-derived growth factor, insulin-like growth factor, transforming growth factor, epidermal growth factor, connective tissue growth factor and fibroblast growth factor; vitamins, particularly vitamin D; minerals, such as calcium, aluminum, strontium, lanthanides (such as lanthanum (III) compounds as described and used in U.S. Pat. No. 7,078,059, incorporated herein by reference) and fluoride; isoflavones, such as ipriflavone; statin drugs, including pravastatin, fluvastatin, simvastatin, lovastatin and atorvastatin; agonsists or antagonist of receptors on the surface of osteoblasts and osteoclasts, including parathormone receptors, estrogen receptors and prostaglandin receptors; bisphosphonate and anabolic bone agents. In one embodiment, vitamin D, calcium, or both are concurrently administered with the pharmaceutical formulations of the present invention.

Generally, preferred embodiments of PTH peptide analogues include those that when administered result in reduced phospholipase-C activity, reduced ability to stimulate bone resorption, and reduced hypercalcemia levels. As defined in the Definitions section herein, “reduced phospholipase-C activity” refers to a PTH peptide analogue that has been truncated or modified in some manner so as to trigger less than full activation of phospholipase-C, as compared to the full-length PTH peptide or other PTH peptide analogues; “reduced bone resorption” refers to a PTH peptide analogue that has been truncated or modified in some manner so as to trigger less bone resorption, as compared to the full-length PTH peptide or other PTH peptide analogues, and “reduced hypercalcemia levels” refers to a PTH peptide analogue that has been truncated or modified in some manner so as to trigger less incidences of hypercalcemia, or lower severity of hypercalcemia, as compared to the full-length PTH peptide or other PTH peptide analogues.

The preferred PTH analogues administered in the methods described herein include [Leu²⁷]cyclo[Glu²²-Lys²⁶]-PTH-(1-31)-NH₂, such as advanced by Zelos Therapeutics, Inc. under the tradename OSTABOLIN-C™ and PTH-(1-31)-NH₂, such as advanced by Zelos Therapeutics, Inc. under the tradename OSTABOLIN™. In another embodiment of the invention, [Leu²⁷]cyclo[Glu²²-Lys²⁶]-PTH-(1-30)-NH₂ is used in the methods described herein. In another embodiment, the hormone can be the linear analogue PTH(1-31), which can have a free carboxyl ending, or be amidated, at the C-terminus. In yet another embodiment, the hormone can be PTH(1-30), which can have a free carboxyl ending, or be amidated, at the C-terminus; or [Leu²⁷]-PTH(1-30)-NH₂. Suitable stabilized solutions of these and other PTH analogues that can be employed in the present methods are described in U.S. Pat. Nos. 5,556,940; 5,955,425; 6,541,450; 6,316,410; and 6,110,892 incorporated herein by reference.

The pharmaceutical compositions and formulations described herein, and in the doses and routes of administration described in detail below, further operate to induce bone formation by stimulating osteoblast differentiation in trabecular and cortical bone while simultaneously reducing the incidence of hypercalcemia (i.e., higher than normal levels of calcium in the blood).

In another aspect of the invention, methods for treating a bone fracture in a subject are provided. The method can include administering to a subject in need thereof a daily dose of a pharmaceutically acceptable formulation of a PTH peptide analogue, wherein the peptide analogue has reduced phospholipase-C activity and maintains adenylate cyclase activity, and wherein the PTH peptide analogue induces bone formation.

The pharmaceutical formulations described herein can be used to heal a fracture in any bone of the subject's skeleton. In preferred embodiments, the pharmaceutical formulations of the present invention are used to heal fractures of the hip, forearm, humerus, wrist, radius, ankle, rib, femur, tibia, and foot. The fractures can be of multiple types as discussed above, and healing can simultaneously occur in a plurality of bones that may be fractured.

In another aspect, the invention provides methods for inducing bone formation in trabecular and cortical bones, as measured by an increase in BMD by administering to a subject in need thereof a daily dose of a pharmaceutically acceptable formulation of a PTH peptide analogue, wherein the peptide analogue has reduced phospholipase-C activity and maintains adenylate cyclase activity.

In preferred embodiments, the pharmaceutical formulations can be used to induce bone formation at the spine, skull, ribs, hips, ankle, and wrists, although any bone of the subject's skeleton can be induced to form bone. In another embodiment, following administration of the PTH pharmaceutical formulations of the present invention, the incidences in the patient population in which the level of serum calcium is above normal is less than the those seen with administration of prior art PTH peptides.

In yet another aspect, the present invention provides methods of treating or preventing renal osteodystrophy (ROD) and related disorders by administering to a subject in need thereof a daily dose of a pharmaceutically acceptable formulation of a PTH peptide analogue, wherein the peptide analogue has reduced phospholipase-C activity and maintains adenylate cyclase activity.

In an embodiment, ROD related disorders are osteitis fibrosa cystica and adynamic bone disease.

Unexpected Results

The pharmaceutical compositions and formulations described herein, and in the doses and routes of administration described in detail below, operate to induce bone formation by stimulating osteoblast differentiation in trabecular and cortical bone while simultaneously reducing or inhibiting osteoclast differentiation, and thus, bone resorption. PTH analogues less than 34 amino acids in length are preferred, because these truncated forms maintain the positive effects of increased bone formation, while minimizing the negative effects of increased bone resorption. Minimizing the bone resorption also leads to less cortical porosity. Administration of the PTH analogues of the present invention at a variety of doses has led to unexpected and superior results when compared to administration of prior PTH analogues. When administered over a course of four months, the PTH analogues of the present invention have been shown to have a similar or greater effect on the increase in BMD of lumbar spine, hip, femoral neck, and trochanter as compared to prior art PTH analogues which are at least 34 amino acid residues in length given over at least a course of a year. For results of prior art PTH analogues, see Neer, N. Eng. J. Med, Vol 344, No. 19, May 2001, p. 1434-1441. These unexpected results are described in detail in the Examples and the Figures.

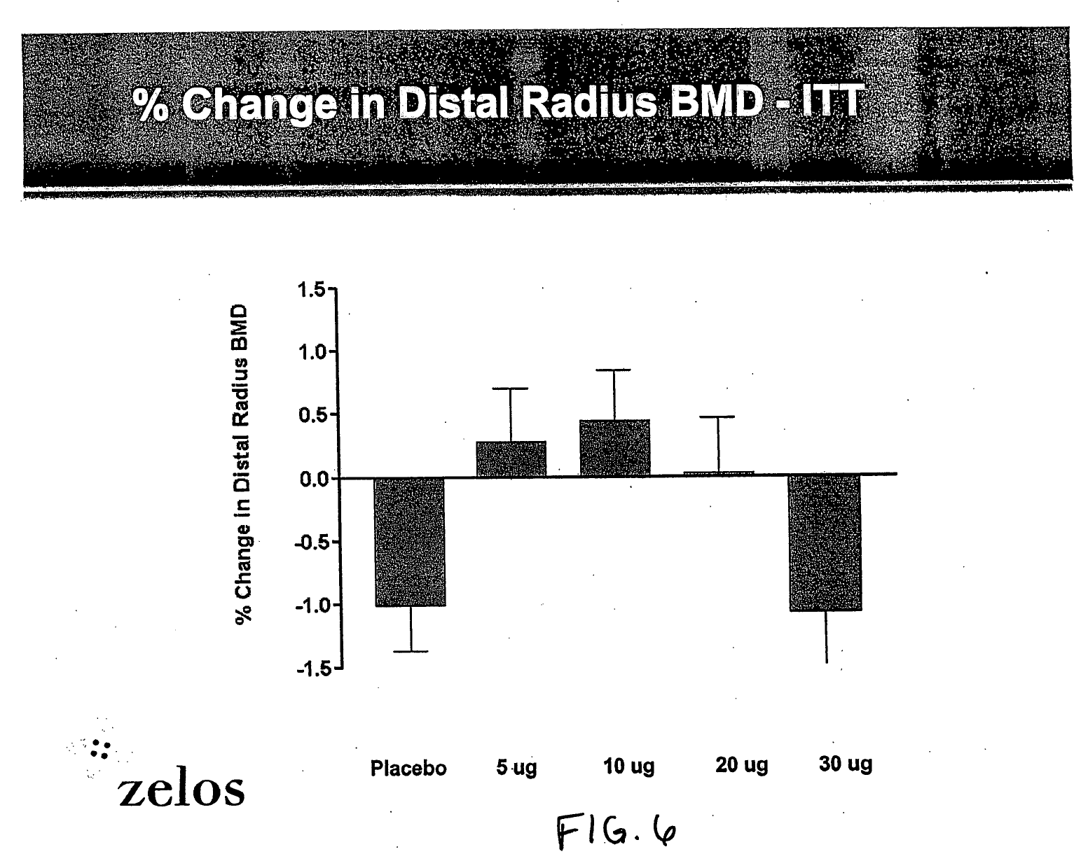

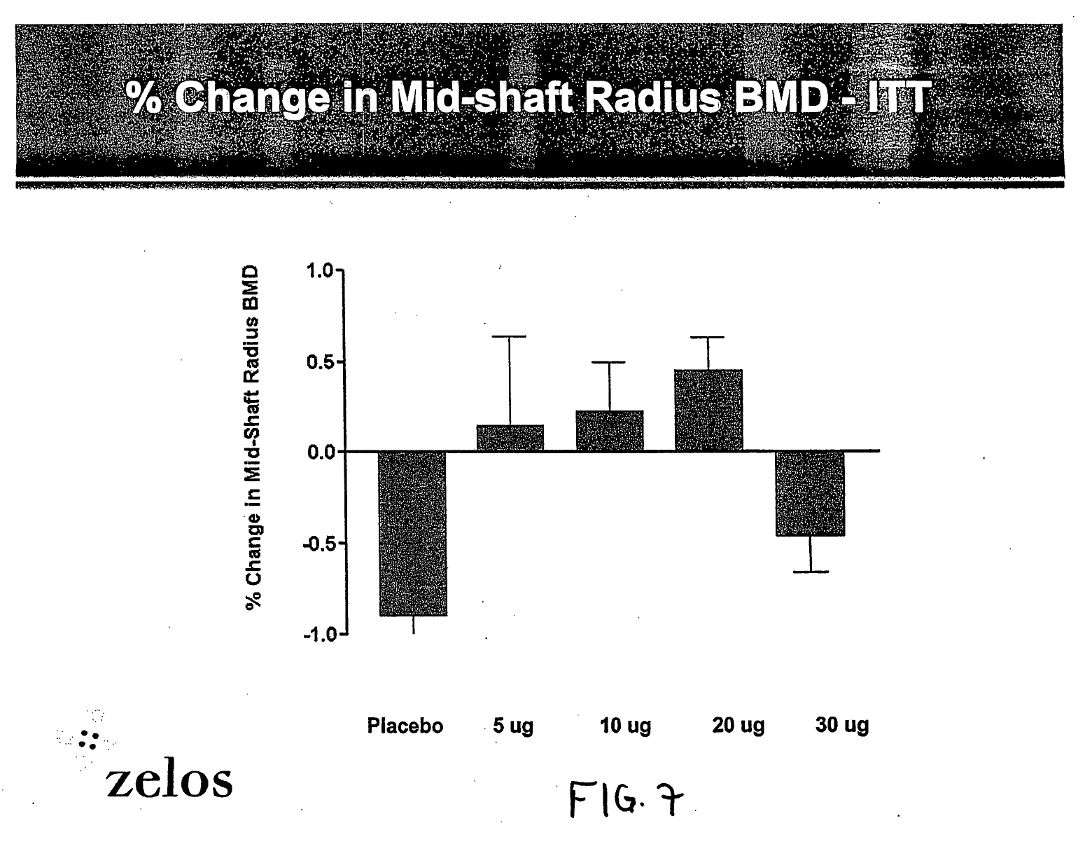

Administration of the peptides of the present invention also has a positive effect on cortical bone, specifically the wrist (the distal and mid-shaft radius, FIGS. 6 and 7). Historically, PTH has been known to increase bone resorption, which increases cortical porosity, thus making it difficult for PTH to increase BMD in cortical bone. The dosages and formulations of the present invention have a positive effect on cortical bone growth as compared to both placebo and to teriparatide, Forteo®. This is an unprecedented finding, demonstrating a statistically significant difference from placebo for 3 active doses.

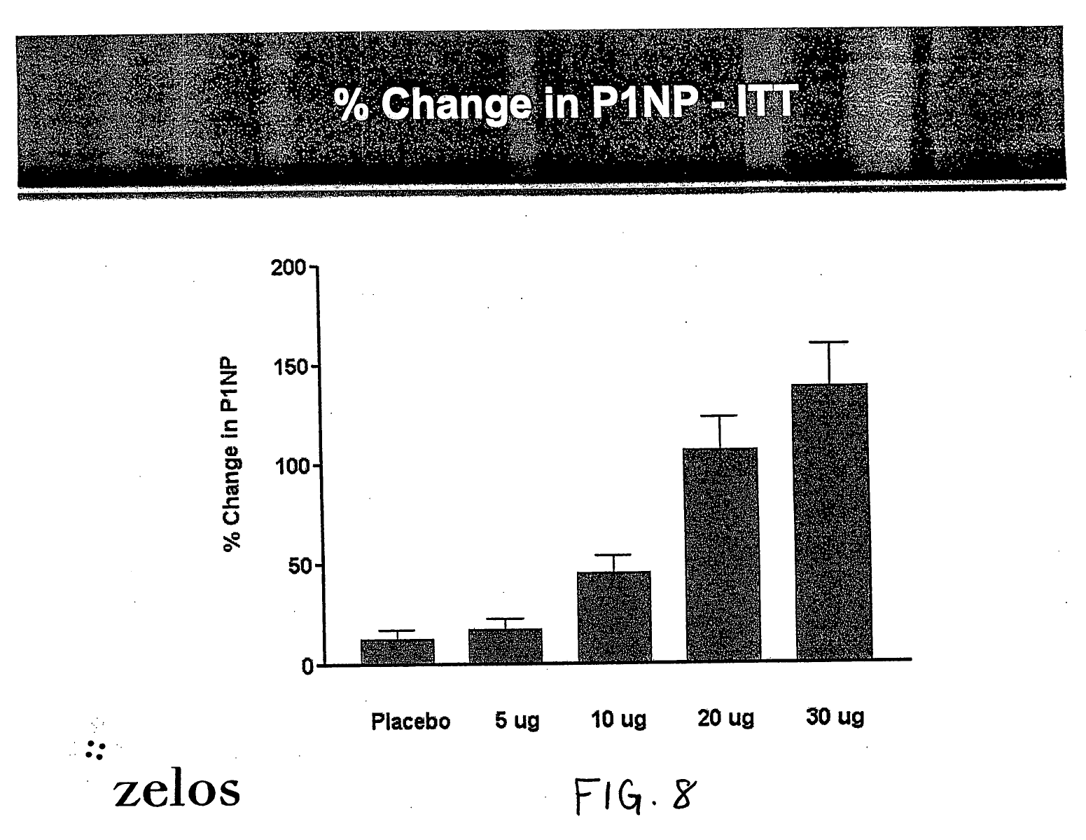

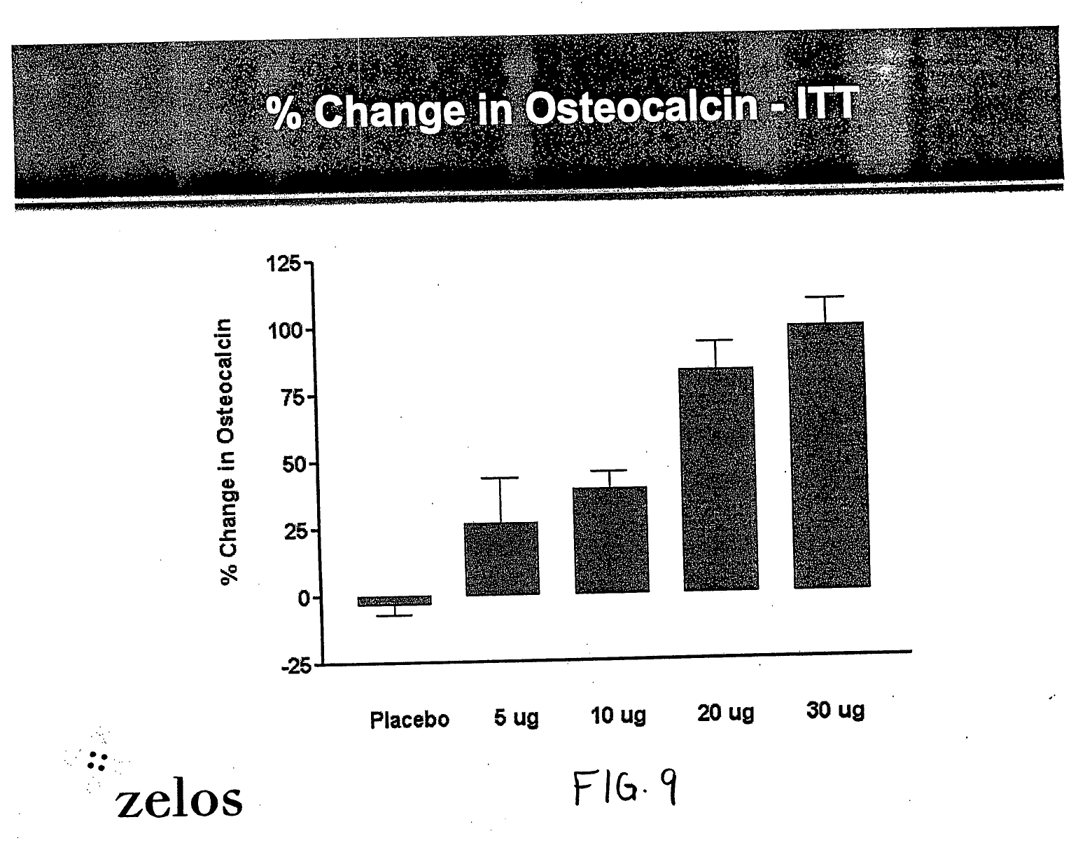

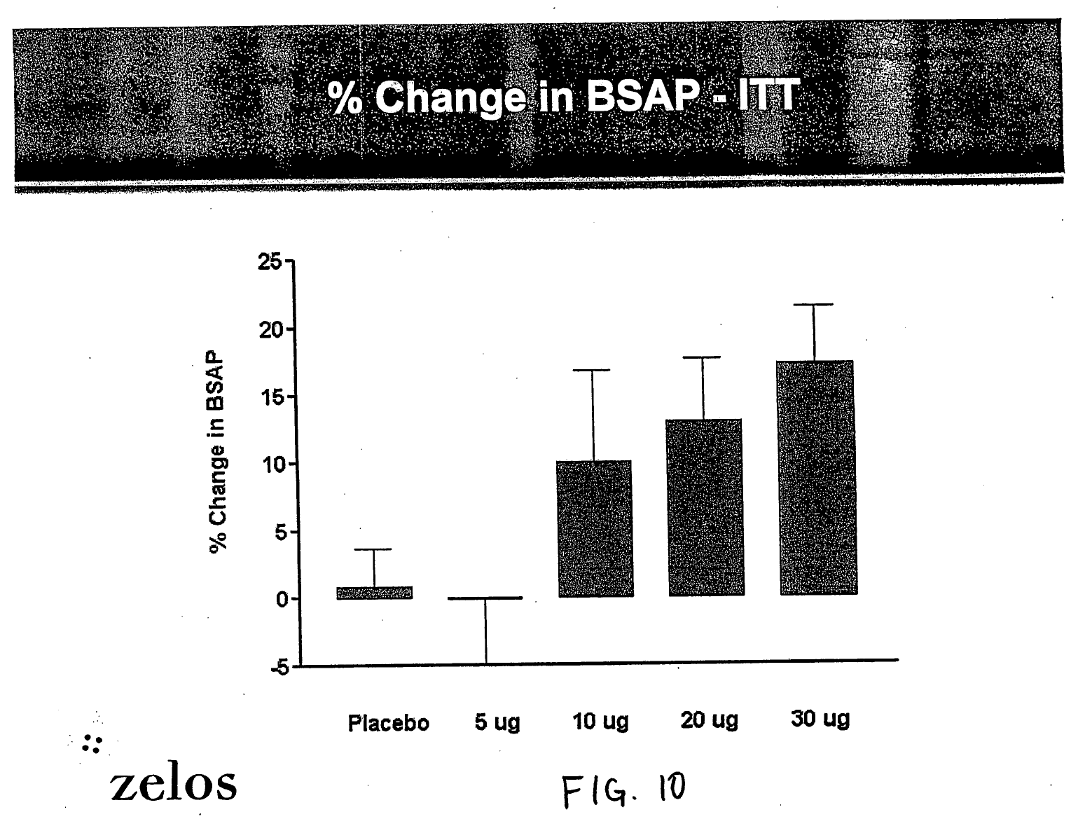

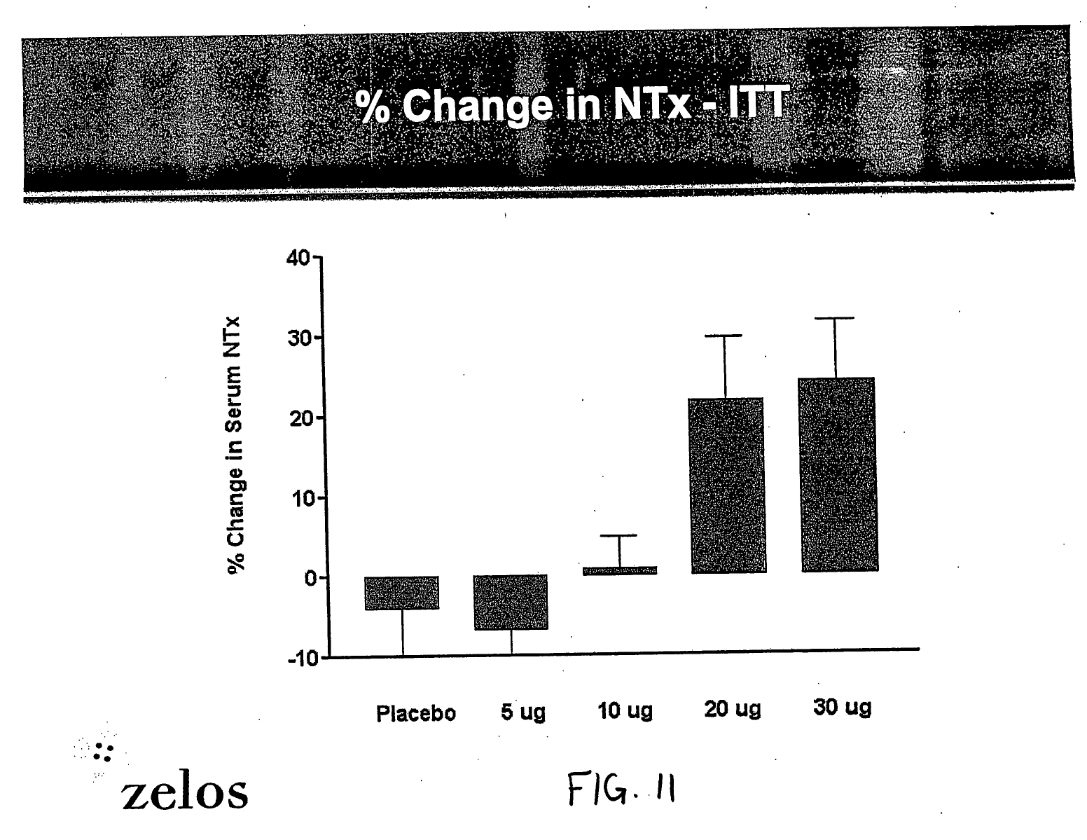

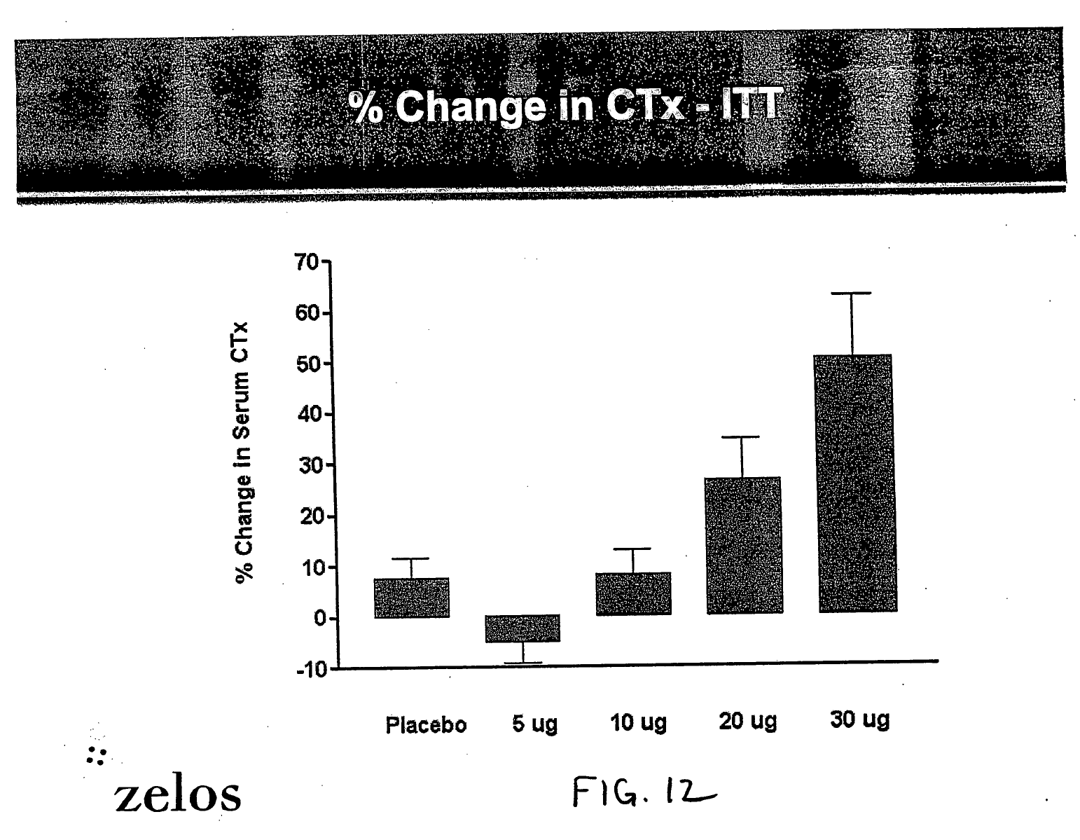

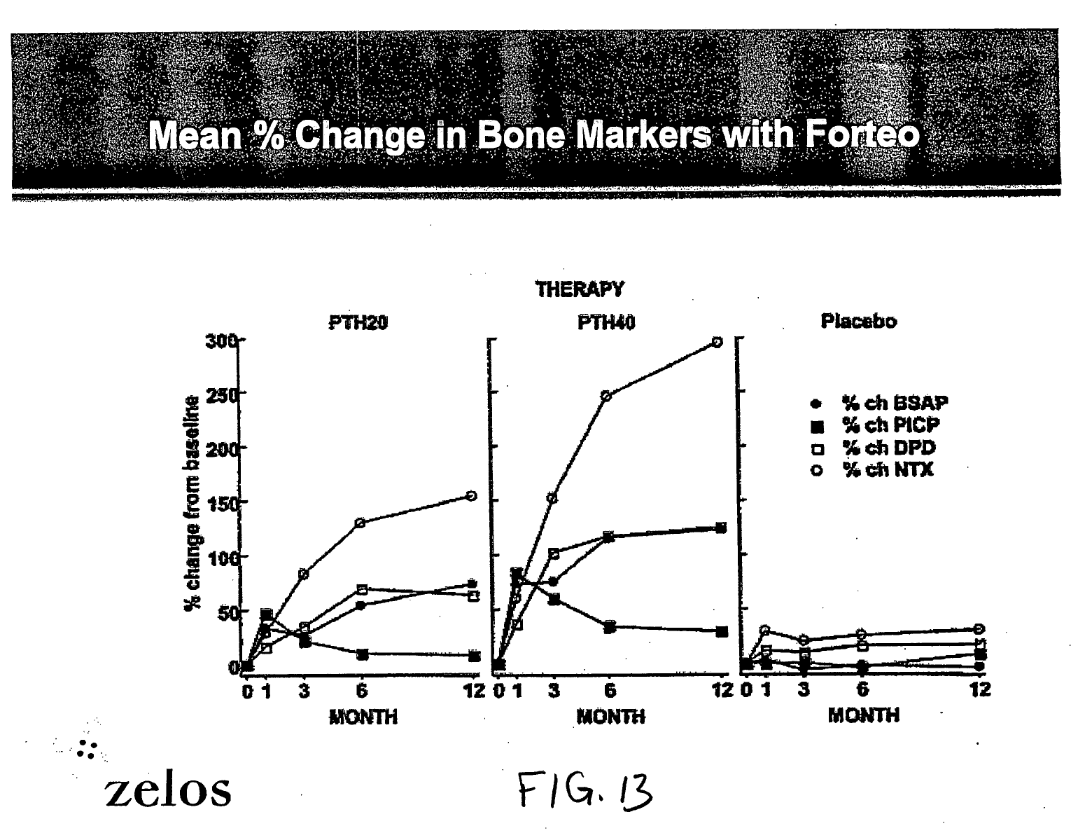

Administration of the PTHs of the present invention also have unexpected results on bone formation and bone resorption markers. The bone formation markers include P1NP, osteocalcin, and BSAP and the bone resorption markers include NTx and CTx. As compared to placebo, the bone formation markers have a greater % change when Ostabolin-C™ is administered at 10, 20, and 30 μg. FIGS. 8-10. There is a robust effect in the increase in the bone formation markers when Ostabolin-C™ is administered at 20 and 30 μg. The bone resorption markers in FIGS. 11-13 demonstrate that although there is some increase in bone resorption following the administration of Ostabolin-C™, this increase is less than that which follows administration of the prior art teriparatide, Forteo® PTH. Neer et al., 2001.

Administration of the PTH peptides of the present invention has also been shown to unexpectedly result in a much lower incidence and severity of hypercalcemia as compared to PTHs known in the art. Hypercalcemia for a patient being administered the PTH peptides means the occurrence of at least one serum calcium value for the patient above the upper limit of normal (2.64 mmol/L; 10.6 mg/dL). Neer et al., 2001.

Administration of Forteo® resulted in an increased level of incidences of hypercalcemia as compared to placebo. FDA approval of Forteo® was based on the results of treatment of 1637 postmenopausal women (with prior vertebral fractures) with 20 or 40 μg/day of Forteo® for an average of 19 months. See Forteo® package insert, incorporated by reference in its entirety, and Neer. While the medication was generally well-tolerated, hypercalcemia was seen at least once in 11% of the 20 μg group subjects and in 28% of the 40 μg group subjects as compared with 2% in the placebo group. In contrast, the administration of low doses of the PTH peptides of the present invention (5, 10, and 20 μg) resulted in only a negligible increase in the incidences of hypercalcemia as compared to placebo. As an example, hypercalcemia was seen at least once in 5% of the placebo group and in 5% of the group being administered 20 μg doses, resulting in no net increase of hypercalcemia. This is in comparison to the 11% seen with Forteo®administered at 20 μg.

Accordingly, administration of the PTH analogues of the present invention at a variety of doses leads to following unexpected results: 1) similar or greater effect on the increase in BMD of lumbar spine, hip, femoral neck, and trochanter when given over a course of only four months as compared to prior art PTH analogues given over a course of at least a year; 2) increase in BMD on cortical bone, specifically the wrist (the distal and mid-shaft radius), whereas prior art PTH peptides have resulted in decease in BMD of cortical bone; and 3) lower amount of incidences and severity of hypercalcemia as compared to prior art PTH peptides.

The PTH peptides of the present invention offer substantial improvements over currently available therapy, as they are an anabolic agent that lead to much lower incidences and severity of hypercalcemia. Based on preclinical and clinical experience to date, the present PTH peptides are a safe and highly effective anabolic agent for treating osteoporosis, without inducing hypercalcemia. Due to its reduced impact on bone resorption, the present PTH peptides also have an improved clinical profile with respect to its effects on bone quality.

The decrease in bone resorption can be measured by a reduction in the level of bone resorptive markers. Although biochemical markers of bone turnover cannot reveal how much bone is present in the skeleton at any given time, and thus, cannot be used to diagnosis osteoporosis or to tell how severe the disease may be, biochemical markers can be used in conjunction with the pharmaceutical compositions and formulations of the present invention to (1) predict bone loss in peri- and post-menopausal women and to (2) monitor the skeletal response to treatment. Unlike bone mineral density (BMD) measurements, biochemical markers are able to detect acute changes in bone turnover. While BMD tests typically detect bone density changes in years, markers are able to detect changes in bone metabolism in weeks or months. Bone turnover can be assessed via the measurement of various biochemical markers. There are two basic types of markers: markers of bone formation and markers of bone resorption. Additionally, these markers can be categorized into two groups: markers that measure substances released by osteoblasts and osteoclasts and markers that measure substances produced during the formation or breakdown of collagen, a primary protein found in bone. As bone remodeling occurs, these substances are released into the blood and, eventually, excreted in the urine. Many biochemical markers can be detected and measured in both the blood (serum) and urine.

The most commonly used assays for bone formation are serum tests of bone-specific alkaline phosphatase (BSAP), osteocalcin and procollagen peptides, proteins produced by osteoblasts and released into the bloodstream during bone formation. Bone resorption markers typically measure the breakdown of products of collagen, the major protein of bone. These include pyridinoline, deoxypyridinoline, urinary deoxypyridinoline (urinary DPD), N-telopeptides (NTX) and C-telopeptides (CTX) of Type I collagen crosslinks.

Earlier assays, such as total alkaline phosphatase and hydroxyproline, are still used in monitoring such metabolic bone diseases as Paget's disease. However, these tests are not sensitive enough to be used in monitoring the more subtle bone remodeling changes that tend to occur in osteoporosis, as levels tend to be within normal limits in individuals with the disease.

An additional unexpected result is the lack of occurrence of osteosarcoma formation with long term administration of the PTH peptides of the present invention. In its packaging, the prior art Forteo® includes a warning label that Forteo® caused an increase in incidence of osteosarcoma in rats. The label warns that Forteo® should not be prescribed for patients who are at increased baseline risk for osteosarcoma. In contrast, the risk of osteosarcoma occurrence with the long term use of the PTH peptides of the present invention is minimal. The present PTH peptides may have no, or less, incidence of osteosarcoma based on a different sequence and different signaling as compared to PTH (1-34). The phospholipase-C and downstream protein kinase C activity, which are minimized with administration of the PTH peptides of the present invention, may be involved in ostoeoblast growth.

Another unexpected result with the PTH peptides of the present invention is the lack of need to monitor serum calcium levels in patients taking these peptides for possible occurrences of hypercalcemia. Serum calcium levels in patients taking the prior art Forteo® is monitored through samples of blood and/or urine during the course of treatment. The Forteo® package insert warns that administration of Forteo® may “exacerbate hypercalcemia.” Use of Forteo® is not recommended for patients with high amounts of calcium in their blood (hypercalcemia), bone cancer or other bone disorders. In contrast, administration of the PTH peptides of the present invention leads to lower incidences of hypercalcemia, as compared to administration of Forteo®. Accordingly, calcium monitoring may not be required with administration of the PTH peptides of the present invention.

Pharmaceutical Compositions/Formulations, Dosing, and Administration

A range of PTH peptide analogue compounds can be used in the methods and compositions of the present invention. Generally, preferred embodiments of PTH peptide analogues include those that when administered result in reduced phospholipase-C activity, reduced bone resorption, and reduced hypercalcemia levels. As defined in the Definitions section herein, “reduced phospholipase-C activity” refers to a PTH peptide analogue that has been truncated or modified in some manner so as to trigger less than full activation of phospholipase-C, as compared to the full-length PTH peptide or other PTH peptide analogues which are at least 34 amino acid residues; “reduced bone resorption” refers to a PTH peptide analogue that has been truncated or modified in some manner so as to trigger less bone resorption, as compared to the full-length PTH peptide or other PTH peptide analogues which are at least 34 amino acid residues, and “reduced hypercalcemia levels” refers to a PTH peptide analogue that has been truncated or modified in some manner so as to trigger less incidences of hypercalcemia, or lower severity of hypercalcemia, as compared to the full-length PTH peptide or other PTH peptide analogues which are at least 34 amino acid residues.

The preferred PTH analogues administered in the methods described herein include [Leu²⁷]cyclo[Glu²²-Lys²⁶]-PTH-(1-31)-NH₂, such as advanced by Zelos Therapeutics, Inc. under the tradename OSTABOLIN-C™ and PTH-(1-31)-NH₂, such as advanced by Zelos Therapeutics, Inc. under the tradename OSTABOLIN™. In another embodiment of the invention, [Leu²⁷]cyclo[Glu²²-Lys²⁶]-PTH-(1-30)-NH₂ is used in the methods described herein. In another embodiment, the hormone can be the linear analogue PTH(1-31), which can have a free carboxyl ending, or be amidated, at the C-terminus. In yet another embodiment, the hormone can be PTH(1-30), which can have a free carboxyl ending, or be amidated, at the C-terminus; or [Leu²⁷]-PTH(1-30)-NH₂. Suitable stabilized solutions of the PTH peptide analogues that can be employed in the present methods are described in U.S. Pat. Nos. 5,556,940; 5,955,425; 6,541,450; 6,316,410; and 6,110,892 incorporated herein by reference.

Dosages

An effective amount of a PTH peptide analogue for use in the present invention is an amount that will provide the desired benefit or therapeutic effect upon administration according to the prescribed regimen. Nonlimiting examples of an effective amount of PTH analogue may range from about 2 μg/day to about 60 μg/day, preferably from about 5 μg/day to about 40 μg/day, more preferably from about 10 μg/day to about 20 μg/day, and more preferably 5, 10, 15, 20, 25, 30, or 35 μg/day. Additional preferred dosages include dosages of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 μg/day. Additional examples of an effective amount of PTH analogue may range from about 14 μg/week to about 420 μg/week, preferably from about 35 μg/week to about 280 μg/week, more preferably from about 70 μg/week to about 140 μg/week, and more preferably 35, 70, 105, 140, 175, 205, or 245 μg/week. The dosages can be administered every day, every two days, every three days, every four days, every five days, every six days, or every seven days (once/week). These dosages can also be adjusted to correct for bioavailability. The doses can also be measured in mmol, taking into account the molecular weight of the PTH peptides used.

The dose may also be selected to provide an effective plasma concentration of PTH analogue. Examples of an effective maximum plasma concentration of PTH peptide analogue concentration may range from about 10 pg/mL to about 400 pg/mL, preferably from about 20 pg/mL to about 300 pg/mL; from about 50 pg/mL to about 280 μg/mL; from about 80 pg/mL to about 250 pg/mL; from about 100 pg/mL to about 150 pg/mL. Other suitable dosage ranges for maximum plasma concentration of PTH peptide analogues include 20-40 pg/mL, 40-60 pg/mL, 60-80 pg/mL, 80-100 pg/mL, 100-120 pg/mL, 120-140 pg/mL, 140-160 pg/mL, 160-180 pg/mL, 180-200 pg/mL, 200-230 pg/mL, 230-260 pg/mL, 260-300 pg/mL, 300-350 pg/mL, and 350-400 pg/mL.

In another specific embodiment of the invention, the peptide is administered in an effective amount that results in the value for area under the curve (herein referred to as “AUC”) in the plasma peptide concentration versus time curve in the range of 5 pg·h/mL-400 pg·h/mL. More preferably, the range of AUC is between 10 pg·h/mL-350 pg·h/mL. More preferably, AUC is in the range of 20 pg·h/mL-300 pg·h/mL. More preferably, AUC is in the range of 50 pg·h/mL-250 pg·h/mL. More preferably, AUC is in the range of 70 pg·h/mL-200 pg·h/mL. More preferably, AUC is in the range of 90 pg·h/mL-150 pg·h/mL.

Even more preferably, AUC is in the range of 95 pg·h/mL-125 pg·h/mL. Other suitable range for AUC is 5 pg·h/mL-20 pg·h/mL, 20 pg·h/mL-50 pg·h/mL, 50 pg·h/mL-70 pg·h/mL, 70 pg·h/mL-90 pg·h/mL, 90 pg·h/mL-100 pg·h/mL, 100 pg·h/mL-110 pg·h/mL, 110 pg·h/mL-120 pg·h/mL, 120 pg·h/mL-130 pg·h/mL, 130 pg·h/mL-150 pg·h/mL, 150 pg·h/mL-175 pg·h/mL, 175 pg·h/mL-200 pg·h/mL, 200 pg·h/mL-225 pg·h/mL, 225 pg·h/mL-250 pg·h/mL, 250 pg·h/mL-275 pg·h/mL, 275 pg·h/mL-300 pg·h/mL, 300-350 pg·h/mL, or 350 pg·h/mL-400 pg·h/mL.

Accordingly, in one aspect, the invention provides a pharmaceutical formulation comprising a therapeutically effective amount of a PTH peptide analogue as the active ingredient in a daily dosage range of 2 μg to 60 μg or a weekly dosage range of 14 μg to 420 μg, wherein the PTH peptide analogue has reduced phospholipase-C activity and maintains adenylate cyclase activity, in admixture with a pharmaceutically acceptable excipient, diluent, or carrier, or combinations thereof.

Routes of Administration

Administration of the PTH peptide analogues of the present invention includes both direct administration, including self-administration, and indirect administration, including the act of prescribing a drug. For example, as used herein, a physician who instructs a patient to self-administer a drug and/or provides a patient with a prescription for a drug is administering the drug to the patient.

A variety of administration routes can be used in accordance with the present invention, including oral, topical, transdermal, nasal, pulmonary, transpercutaneous (wherein the skin has been broken either by mechanical or energy means), rectal, buccal, vaginal, via an implanted reservoir, or parenteral. Parenteral includes subcutaneous, intravenous, intramuscular, intraperitoneal, intra-articular, intra-synovial, intrastemal, intrathecal, intrahepatic, intralesional and intracranial injection or infusion techniques. More preferably, the route of administration is subcutaneous administration.

Formulations

A stabilized solution of a parathyroid hormone can include a stabilizing agent, a buffering agent, a preservative, an antibacterial agent and the like. The stabilizing agent incorporated into the solution or composition includes a polyol which includes a saccharide, preferably a monosaccharide or disaccharide, e.g., glucose, trehalose, raffinose, or sucrose; a sugar alcohol such as, for example, mannitol, sorbitol or inositol, and a polyhydric alcohol such as glycerine or propylene glycol or mixtures thereof. A preferred polyol is mannitol or propylene glycol. The concentration of polyol may range from about 1 to about 20 wt-%, preferably about 3 to 10 wt-% of the total solution.

The buffering agent employed in the solution or composition of the present invention may be any acid or salt combination which is pharmaceutically acceptable. Useful buffering systems are, for example, acetate, tartrate or citrate sources. Preferred buffer systems are acetate or tartrate sources, most preferred is an acetate source. The concentration of buffer may be in the range of about 2 mM to about 500 mM, preferably about 2 mM to 100 mM.

The stabilized solution or composition of the present invention may also include a parenterally acceptable preservative. Such preservatives include, for example, cresols, benzyl alcohol, phenol, benzalkonium chloride, benzethonium chloride, chlorobutanol, phenylethyl alcohol, methyl paraben, propyl paraben, thimerosal and phenylmercuric nitrate and acetate. A preferred preservative is m-cresol or benzyl alcohol; most preferred is m-cresol. The amount of preservative employed may range from about 0.1to about 2 wt-%, preferably about 0.3 to about 1.0 wt-% of the total solution.

The parathyroid hormone compositions can, if desired, be provided in a powder form containing not more than 2% water by weight, that results from the freeze-drying of a sterile, aqueous hormone solution prepared by mixing the selected parathyroid hormone, a buffering agent and a stabilizing agent as above described. Especially useful as a buffering agent when preparing lyophilized powders is a tartrate source. Particularly useful stabilizing agents include glycine, sucrose, trehalose and raffinose.

In addition, parathyroid hormone can be formulated with typical buffers and excipients employed in the art to stabilize and solubilize proteins for parenteral administration. Art recognized pharmaceutical carriers and their formulations are described in Martin, “Remington's Pharmaceutical Sciences,” 15th Ed.; Mack Publishing Co., Easton (1975).

The PTH peptide analogue may also be formulated into a composition suitable for administration by any convenient route, e.g., orally (including sublingually), topically, transdermally (including percutaneous absorption of the composition through the skin, such as by patches, ointments, creams, gels, salves and the like), intranasally, rectally or inhaled as a dry powder, aerosol, or mist, for pulmonary delivery.

Such forms of the compounds of the invention may be administered by conventional means for creating aerosols or administering dry powder medications using devices such as for example, metered dose inhalers, nasal sprayers, dry powder inhaler, jet nebulizers, or ultrasonic nebulizers. Such devices optionally may include a mouthpiece fitted around an orifice. It should be understood, however, that the invention embraces all forms of administration which make the PTH peptide analogues systemically or locally available.

In addition to the usual meaning of administering the formulations described herein to any part, tissue or organ whose primary function is gas exchange with the external environment, for purposes of the present invention, “pulmonary” is also meant to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

Orally administrable compositions may, if desired, contain one or more physiologically compatible carriers and/or excipients and may be solid or liquid. Intranasal administration to the subject includes administering a therapeutically effective amount of the PTH peptide analogue to the mucous membranes of the nasal passage or nasal cavity of the subject. Pharmaceutical compositions for nasal administration can include, for example, nasal spray, nasal drops, suspensions, gels, ointments, creams, or powders.

Pharmaceutically acceptable compositions of the peptide described herein can be used according to the method of the present invention. The pharmaceutical compositions described herein can optionally include one or more pharmaceutically acceptable excipients. Such pharmaceutically acceptable excipients are well known in the art and include, for example, salts (such as protamine sulfate, disodium hydrogen phosphate, potassium hydrogen phosphate, sodium chloride, zinc salts, colloidal silica and magnesium trisilicate), surfactant(s), water-soluble polymers (such as polyvinyl pyrrolidone, cellulose based substances, polyethylene glycol, polyacrylates, sodium carboxymethylcellulose, waxes and polyethylene-polyoxypropylene-block polymers), preservatives, antimicrobials, antioxidants, cryo-protectants, wetting agents, viscosity agents, tonicity modifying agents, levigating agents, absorption enhancers, penetration enhancers, pH modifying agents, muco-adhesive agents, coloring agents, flavoring agents, diluting agents, emulsifying agents, suspending agents, solvents, co-solvents, buffers (such as phosphates, glycine, sorbic acid, potassium sorbate and partial glyceride mixtures of saturated vegetable fatty acids), serum proteins (such as human serum albumin), ion exchangers and combinations of these excipients.

The excipient included within the pharmaceutical compositions of the invention is chosen based on the expected route of administration of the composition in therapeutic applications. Accordingly, compositions designed for oral, lingual, sublingual, buccal and intrabuccal administration can be made without undue experimentation by means well known in the art, for example, with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Solid dosage forms, such as tablets, pills and capsules, may also contain one or more binding agents, filling agents, suspending agents, disintegrating agents, lubricants, sweetening agents, flavoring agents, preservatives, buffers, wetting agents, disintegrants, effervescent agents, and other excipients. Such excipients are known in the art. Examples of filling agents are lactose monohydrate, lactose anhydrous, and various starches. Examples of binding agents are various celluloses and cross-linked polyvinylpyrrolidone, microcrystalline cellulose, and silicifized microcrystalline cellulose (SMCC). Suitable lubricants, including agents that act on the flowability of the powder to be compressed, are colloidal silicon dioxide, talc, stearic acid, magnesium stearate, calcium stearate, and silica gel. Examples of sweeteners are any natural or artificial sweetener, such as sucrose, xylitol, sodium saccharin, cyclamate, aspartame, and accsulfame K. Examples of flavoring agents are bubble gum flavor, fruit flavors, and the like. Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride. Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, lactose such as lactose monohydrate, lactose anhydrous, dibasic calcium phosphate, mannitol, starch, sorbitol, sucrose and glucose. Suitable disintegrants include corn starch, potato starch, and modified starches, crosspovidone, sodium starch glycolate, and mixtures thereof. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propyl parabens as preservatives, a dye and a flavoring such as cherry or orange flavor, and the like.

The compositions may take any convenient form including, for example, tablets, coated tablets, capsules, lozenges, aqueous or oily suspensions, solutions, emulsions, syrups, elixirs and dry products suitable for reconstitution with water or another suitable liquid vehicle before use. The compositions may advantageously be prepared in dosage unit form. Tablets and capsules according to the invention may, if desired, contain conventional ingredients such as binding agents, for example syrup, acacia, gelatin, sorbitol, tragacanth or polyvinyl-pyrollidone; fillers, for example lactose, sugar, maize-starch, calcium phosphate, sorbitol or glycine; lubricants, for example magnesium stearate, talc, polyethylene glycol or silica; disintegrants, for example potato starch; or acceptable wetting agents such as sodium lauryl sulphate. Tablets may be coated according to methods well known in the art.

Liquid compositions may contain conventional additives such as suspending agents, for example sorbitol syrup, methyl cellulose, glucose/sugar syrup, gelatin, hydroxymethylcellulose, carboxymethylcellulose, aluminium stearate gel or hydrogenated edible fats; emulsifying agents, for example lecithin, sorbitan monooleate or acacia; non-aqueous vehicles, which may include edible oils, for example vegetable oils such as arachis oil, almond oil, fractionated coconut oil, fish-liver oils, oily esters such as polysorbate 80, propylene glycol, or ethyl alcohol; and preservatives, for example methyl or propyl p-hydroxybenzoates or sorbic acid. Liquid compositions may conveniently be encapsulated in, for example, gelatin to give a product in dosage unit form.

Formulations for oral delivery may be formulated in a delayed release formulation such that the PTH peptide analogue is delivered to the large intestine. Delayed release formulations are well known in the art and include for example, delayed release capsules or time pills, osmotic delivery capsules etc.

Compositions for parenteral administration may be formulated using an injectable liquid carrier such as sterile pyrogen-free water, sterile peroxide-free ethyl oleate, dehydrated alcohol or propylene glycol or a dehydrated alcohol/propylene glycol mixture, and may be injected intravenously, intraperitoneally, subcutaneously or intramuscularly. Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Compositions for rectal administration may be formulated using a conventional suppository base such as cocoa butter or another glyceride.

Compositions for topical administration include ointments, creams, gels, lotions, shampoos, paints, powders (including spray powders), pessaries, tampons, sprays, dips, aerosols, pour-ons and drops. The active ingredient may, for example, be formulated in a hydrophilic or hydrophobic base as appropriate.

It may be advantageous to incorporate an antioxidant, for example ascorbic acid, butylated hydroxyanisole or hydroquinone in the compositions of the invention to enhance their storage life.

Dosing Regimen

Administration in this invention may consist of one or more cycles; during these cycles one or more periods of osteoclastic and osteoblastic activity will occur, as well as one or more periods when there is neither osteoclastic nor osteoblastic activity. Alternatively, administration may be conducted in an uninterrupted regimen; such a regimen may be a long term regimen, e.g., a permanent regimen.

It will be understood that the dosages of compositions and the duration of administration according to the invention will vary depending on the requirements of the particular subject. The precise dosage regime will be determined by the attending physician or veterinary surgeon who will, inter alia, consider factors such as body weight, age and symptoms (if any). The compositions may if desired incorporate one or more further active ingredients.

During the dosing regimen, the hormone can be administered regularly (e.g., once or more each day or week), intermittently (e.g., irregularly during a day or week), or cyclically (e.g., regularly for a period of days or weeks followed by a period without administration). Regular administration can include once daily, once every two days, once every three days, once every four days, once every five days, once every six days, or once every seven days (once/week). Preferably PTH is administered once daily for 1-7 days for a period ranging from 3 months for up to 3 years in osteoporotic patients. In additional embodiments, PTH is administered for no less than 8 days. The present invention also encompasses embodiments wherein PTH is administered on a weekly basis.

Preferably, cyclic administration includes administering a parathyroid hormone for at least 2 bone remodeling cycles and withdrawing parathyroid hormone for at least 1 bone remodeling cycle. Another preferred regime of cyclic administration includes administering the parathyroid hormone for at least about 12 to about 24 months and withdrawing parathyroid hormone for at least 6 months. Typically, the benefits of administration of a parathyroid hormone persist after a period of administration. The benefits of several months of administration can persist for as much as a year or two, or more, without additional administration.

If desired, the PTH peptide analogue compound may be administered simultaneously or sequentially with other active ingredients, e.g., bone enhancing agents. These active ingredients may, for example include other medicaments or compositions capable of interacting with the bone remodelling cycle and/or which are of use in fracture repair. Such medicaments or compositions may, for example, be those of use in the treatment of osteoarthritis or osteoporosis as discussed above.

In yet a further aspect, the invention provides a method of treatment or prevention of bone-related diseases, in particular osteoporosis, which comprises administering to a mammal, including humans, in need of such treatment (a) an effective amount of PTH peptide analogues during a period of approximately 6 to 24 months; and (b) after the administration of PTH has been terminated, an effective amount of a bone resorption inhibitor during a period of approximately 12 to 36 months. The bone resorption inhibitor can be a bisphosphonate, e.g. alendronate; or a substance with estrogen-like effect, e.g. estrogen; or a selective estrogen receptor modulator, e.g. raloxifene, tamoxifene, droloxifene, toremifene, idoxifene, or levormeloxifene; or a calcitonin-like substance, e.g. calcitonin; or a vitamin D analog; or a calcium salt.

For human administration, preparations should meet sterility, pyrogenicity, general safety and purity standards as required by the FDA.

Kits

The present invention also encompasses a kit including the present pharmaceutical compositions and to be used with the methods of the present invention. The kit can contain a vial, for example, which contains a formulation of the present invention and suitable carriers, either dried or in liquid form. The kit further includes instructions in the form of a label on the vial and/or in the form of an insert included in a box in which the vial is packaged, for the use and administration of the compounds. The instructions can also be printed on the box in which the vial is packaged. The instructions contain information such as sufficient dosage and administration information so as to allow a worker in the field to administer the drug. It is anticipated that a worker in the field encompasses any doctor, nurse, or technician who might administer the drug, or a patient who might self-administer the pharmaceutical composition.

In one embodiment the kit contains a medication delivery pen that houses a cartridge assembly containing a vial or cartridge that has the capability of holding about a 60 day supply of daily doses of the pharmaceutical compositions described herein. In additional embodiments, the pen has the capability of holding a 1, 2, 3, 4, 5, 6, 7, or 8 week supply of daily doses of the pharmaceutical compositions described herein. In preferred embodiments, the pen has the capability of holding a 2 or 4 week supply of daily doses of the pharmaceutical compositions described herein. Such a device provides ease of use for self-administration of the pharmaceutical compositions described herein.

In a further embodiment, the cartridge can contain a liquid dosage of the pharmaceutical composition, or a lyophilized dosage, which is reconstituted by the user prior to injection. Those of skill in the pharmaceutical arts will recognize that medication delivery pens, cartridge assemblies for holding a liquid or lyophilized pharmaceutical dosage formulation for same, and methods of lyophilizing and sealing an injectable composition are known in the art, as evidenced by U.S. Pat. Nos. 5,334,162; 6,053,893; and 6,648,859 the teachings of which are incorporated herein by reference.

The examples which follow are illustrative of the invention and are not intended to be limiting.

### EXAMPLE 1

### Synthesis and Purification of [Leu²⁷]cyclo[Glu²²-Lys²⁶]-hPTH-(1-31)-NH₂

This peptide was synthesized and purified as described in U.S. Pat. No. 5,955,425, the teachings of which are incorporated herein by reference, with Lys-Alloc and Glu-OA11 substituted at position 26 and 22, respectively. After the addition of Fmoc-Ser¹⁷, the peptide-resin was removed from the column to a reaction vial (Minivial, Applied Science), suspended in 1.7 ml of a solution of tetrakis(triphenylphosphine)palladium(0) (0.24 mmol), 5% acetic acid and 2.5% N-methylmorpholine (NMM) in dichloromethane (DCM) under argon, then shaken at 20° C. for 6 hr to remove the allyl and alloc protecting groups (Solé, N. A. et al (1993) In Peptides: Chemistry, Structure, and Biology, Smith, J. And Hodges, R. (Eds), ESCOM pp. 93-94, incorporated herein by reference). The peptide resin was then washed with 0.5% diethyldithiocarbamate (DEDT), 0.5% NMM in DMF (50 ml), followed by DMF (50 ml) and DCM (50 ml). The peptide (0.06 mmol) was cyclized by shaking with 0.06 mmol of 1-hydroxy-7-azabenzotriazole (HOAt)/0.12 mmol NMM in 2 ml DMF for 14 h at 20° C. (Carpino, L. A. (1993) J. Am. Chem. Soc. 115, 4397-4398). The peptide-resin was filtered, then washed once with DMF, repacked into the column, and washed with DMF until bubbles were removed from the suspension. The remaining synthesis was carried out as the linear counterpart above except that the N-terminal Fmoc group was not removed. The Fmoc-peptide was cleaved from the resin with reagent K as described above. The HPLC was carried out as the linear counterpart above, with the Fmoc group removed prior to the final HPLC.

Other suitable stabilized solutions of the PTH peptide analogues that can be employed in the present methods can be synthesized and purified as described in U.S. Pat. Nos. 5,556,940; 5,955,425; 6,541,450; 6,316,410; and 6,110,892 the teachings of which are incorporated herein by reference.

### EXAMPLE 2

### [Leu²⁷]cyclo[Glu²²-Lys²⁶]-hPTH-(1-31)-NH₂ Promotes Growth in Both Trabecular and Cortical Bones in a Monkey Model

The peptide [Leu²⁷]cyclo[Glu²²-Lys²⁶]-hPTH-(1-31)-NH₂ Ostabolin-C™ was administered daily by subcutaneous injection to gonad-intact cynomolgus monkeys (4/sex/group) at dose levels of 0, 2, 10 and 25 μg/kg for 52 weeks. Monkeys were 30 to 40 months of age (2.3-3.5 kg) at treatment start. Tibiae were retained for histomorphometry following labeling with calcein green 15 and 5 days prior to euthanasia. Bone mass, as measured by DXA (dual-energy x-ray absorptiometry) and QCT (quantitative computed tomography), was increased at the lumbar spine, femur and tibia. Changes in vertebral BMD (bone mineral density) translated into significant increases in bone strength. The peptide [Leu²⁷]cyclo[Glu22-Lys²⁶]-hPTH-(1-31)-NH₂ substantially increased osseous accretion in the cancellous and endocortical bone compartments of the proximal tibia at all doses. Tibial cancellous bone volume increased by more than 50% in all the peptide [Leu²⁷]cyclo[Glu²²-Lys²⁶]-hPTH-(1-31)-NH₂ treated groups compared to controls and in the tibial mid-diaphysis, increases in cortical width and relative cortical area with concurrent decreases in medullary area were observed. Only minor increases in cortical porosity were observed at the two highest dose levels. The increase in bone mass appeared to be related to increases in bone formation and decreases in bone resorption as measured by a significant reduction in osteoclast surface. Increases in indices of bone formation were associated with decreases in indices of bone resorption (decreased bone resorption markers, decreased osteoclast surface area, minimal cortical porosity), consistent with the uncoupling of these events. This combination of anabolic and anti-catabolic actions may have significant therapeutic value in the treatment of osteoporosis.

### EXAMPLE 3

### Pre-Clinical Cortical Porosity Data

Comparative data regarding increase in cortical bone porosity in monkey subjects using Ostabolin C at a variety of doses and using the prior art PTHs 1-34 is shown below.

| | | | | | % Cortical | Study |
|---|---|---|---|---|---|---|
| Molecule | Model | Site | M/F | Dose | Porosity | Reference |
| Ostabolin-C | Gonad | Tibial | M | Control | 3.4 ± 0.89 | Zelos |
| | intact young | Mid- | | 2 μg/kg/day | 4.2 ± 0.29 | |
| | Cynomolgus monkeys | Diaphysis | | 10 μg/kg/day | 5.1 ± 1.08 | |
| | treated daily | | | 25 μg/kg/day | 8.0 ± 5.54 | |
| | for 12 months | | | | | |
| | Gonad | Tibial | F | Control | 2.0 ± 0.32 | Zelos |
| | intact young | Mid- | | 2 μg/kg/day | 2.5 ± 0.41 | |
| | Cynomolgus monkeys | Diaphysis | | 10 μg/kg/day | 2.6 ± 0.85 | |
| | treated daily | | | 25 μg/kg/day | 3.2 ± 0.87 | |
| | for 12 months | | | | | |
| Ostabolin-C | Gonad | Tibial | M | Control | 3.5 ± 1.18 | Zelos |
| | intact young | Mid- | | 10 μg/kg/day | 3.7 ± 0.70 | |
| | Cynomolgus monkeys | Diaphysis | | 25 μg/kg/day | 5.8 ± 1.82 | |
| | treated dilay | | | 16.4 ± 7.14* | | |
| | for 6 weeks | | | 80 μg/kg/day | | |
| | Gonad | Tibial | F | Control | 3.3 ± 0.90 | Zelos |
| | intact young | Mid- | | 10 μg/kg/day | 3.2 ± 0.97 | |
| | Cynomolgus monkeys | Diaphysis | | 25 μg/kg/day | 4.0 ± 1.25 | |
| | treated dilay | | | | 10.6 ± 0.35 | |
| | for 6 weeks | | | 80 μg/kg/day | | |
| PTH 1-34 | OVX adult | Humerus | F | Control | ˜5.0 | Burr et al., |
| | Cynomolgus monkeys | Mid- | | 1 μg/kg/day | ˜15.0* | JBMR 16: |
| | treated daily | Diaphysis | | 5 μg/kg/day | ˜25* | 157-165, 2001 |
| | for 18 months | | | | | |
| | OVX adult | Femoral | F | Control | 6.7 ± 0.7 | Sato et al., |
| | Cynomolgus monkeys | Neck | | 1 μg/kg/day | 8.5 ± 0.8* | JBMR 19: |
| | treated daily | | | 5 μg/kg/day | 8.9 ± 0.6* | 623-629, 2004 |
| | for 18 months | | | | | |

### EXAMPLE 4

### Pre-Clinical Toxicity Data

The below table demonstrates that the prior art PTH, 1-34, teriparatide, Forteo®, is more nephrotoxic than Ostabolin-C™, the difference possibly being linked to the different hypercalcemic states. As shown below, PTH-(1-34) induces a mineralizing nephropathy in monkeys and possibly rats. A NoAEL was not established for the monkey. Ostabolin-C™ was nephrotoxic only in monkeys and a NoAEL was established. Ostabolin-C™ is at least 4-fold safer than PTH-(1-34).

| TERIPARATIDE, FORTEO ® | | | OSTABOLIN C | | |
|---|---|---|---|---|---|
| | Doses | | Doses | | |
| Study | μg/kg | Results | μg/kg | Results | DIFFERENCES |
| Toxicity, 12 | 0, 0.5, | Free Ca increased all | 0, 2, | Variable free Ca: | Ostabolin-C not |
| mth monkey | 2, 10 | doses; tubulo-interstitial | 10, 25 | increased week 31, | hypercalaemic |
| | | nephritis all doses; | | decreased week 52. | and > 4-fold less |
| | | serum neutralising | | tubulo-interstitial | nephrotoxic than |
| | | antibodies detected all | | nephritis mid and high | PTH-(1-34) |
| | | doses most frequently | | dose. Bone hypertrophy | |
| | | high dose at wk 50 | | all doses. NoAEL 2 μg/kg | |
| | | NoAEL <0.5 μg/kg | | | |

### EXAMPLE 5

### Clinical Study of OSTABOLIN-C™

A four month Phase II clinical study was undertaken to investigate the safety, tolerability and efficacy of Ostabolin-C™ in post-menopausal women with low bone mineral density (BMD). Comparative data from this study demonstrates that the use of Ostabolin-C™ has many advantages over the current therapy, use of 1-34 PTH, teriparatide, Forteo®. The clinical protocol is a 16-week phase II randomized, double-blind, placebo-controlled, parallel group, dose finding study to investigate the safety, tolerability and efficacy of Ostabolin-C™ in post-menopausal women with low bone mineral density (BMD). In this study, 261 patients underwent four months of daily dosing of placebo and four active groups. The active groups included daily administration of Ostabolin-C™ in doses of 5, 10, 20, and 30 μg. Ostabolin-C™ is formulated as a clear, colorless liquid provided in pre-filled syringes and injected subcutaneously (SC). Subjects self-administer SC 0.1 mL injections of their assigned dose of Ostabolin-C™ 5, 10, 20 or 30 μg or placebo daily for 16 weeks in rotating quadrants of the abdomen. The subjects were post-menopausal women (for at least 5 years) with moderate osteoporosis.

The key endpoints for the study include change in mean BMD at the lumbar spine, as assessed by dual energy x-ray absorptiometry (DEXA), and measured by change from the Baseline visit. The Baseline visit is the first visit of the patient, before undergoing any treatment. Secondary efficacy endpoints include the following, as measured by change from Baseline visit:

| | |
|---|---|
| DEXA: | Bone formation and resorption markers: |
| Mean femoral neck BMD | Serum osteocalcin |
| Mean trochanter BMD | Serum amino terminal pro-peptide of |
| Mean total hip BMD | type 1 pro-collagen (P1NP) |
| Mean radial BMD (distal and | Bone specific alkaline phosphatase |
| midshaft) | (BSAP) |
| Bone mineral content (BMC) | Serum C-telopeptide (CTx) |
| Bone area | Serum N-telopeptide (NTx) |

Other measurements:
Lateral thoracic, lumbar spine and left antero-posterior hip radiographs
Height

### EXAMPLE 6

### Clinical Results—Effects of Low Dose (5, 10, and 20 μg) Ostabolin-C™

Administration of a daily dosage of 5, 10, and 20 μg of Ostabolin-C™ as described above in Example 5 demonstrates robust bone anabolic effects at multiple sites in the body, including the spine, the hip, and the wrist without the concomitant negative effects previously seen with the use of prior art PTHs. The unprecedented BMD increases at the mid-radius and the lower incidence and severity of hypercalcemia make these highly attractive doses.

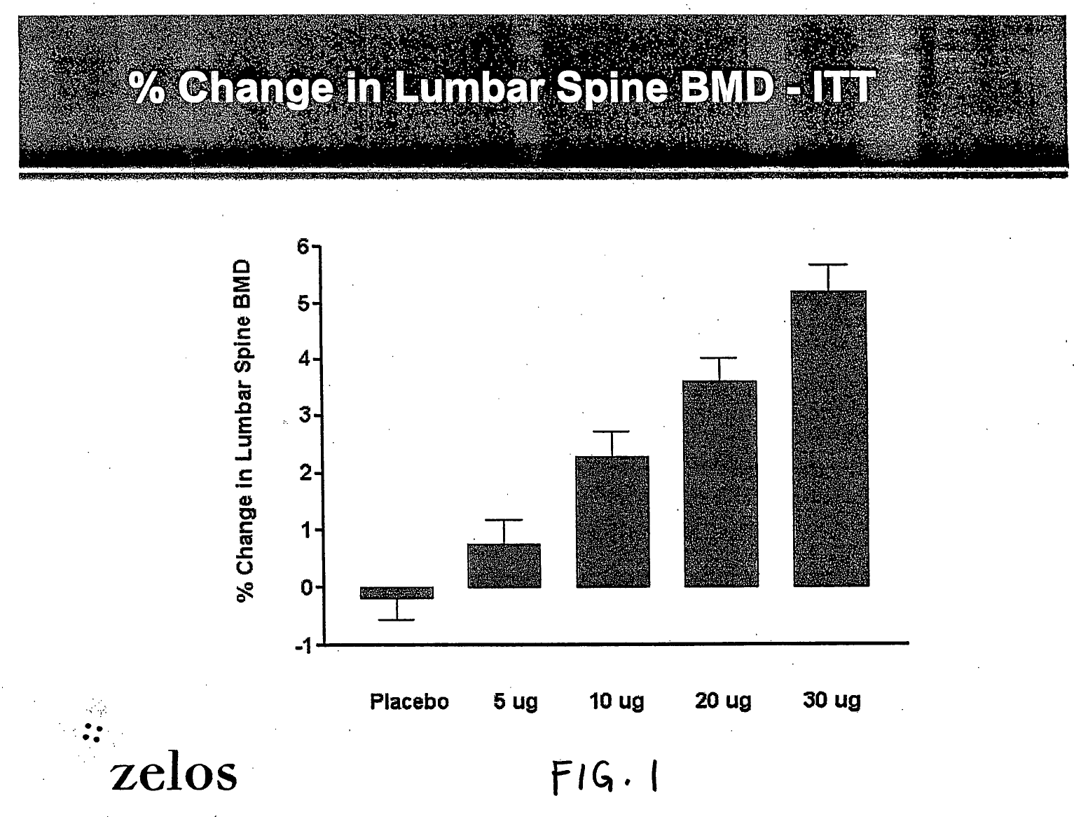

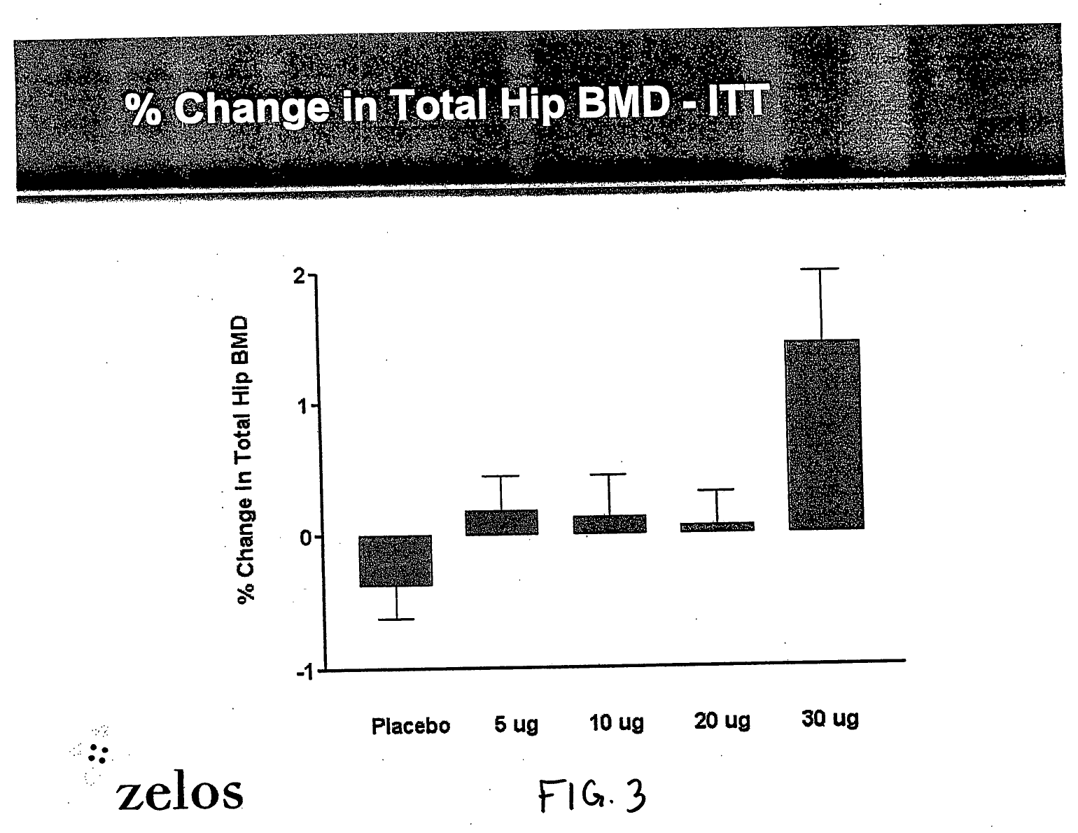

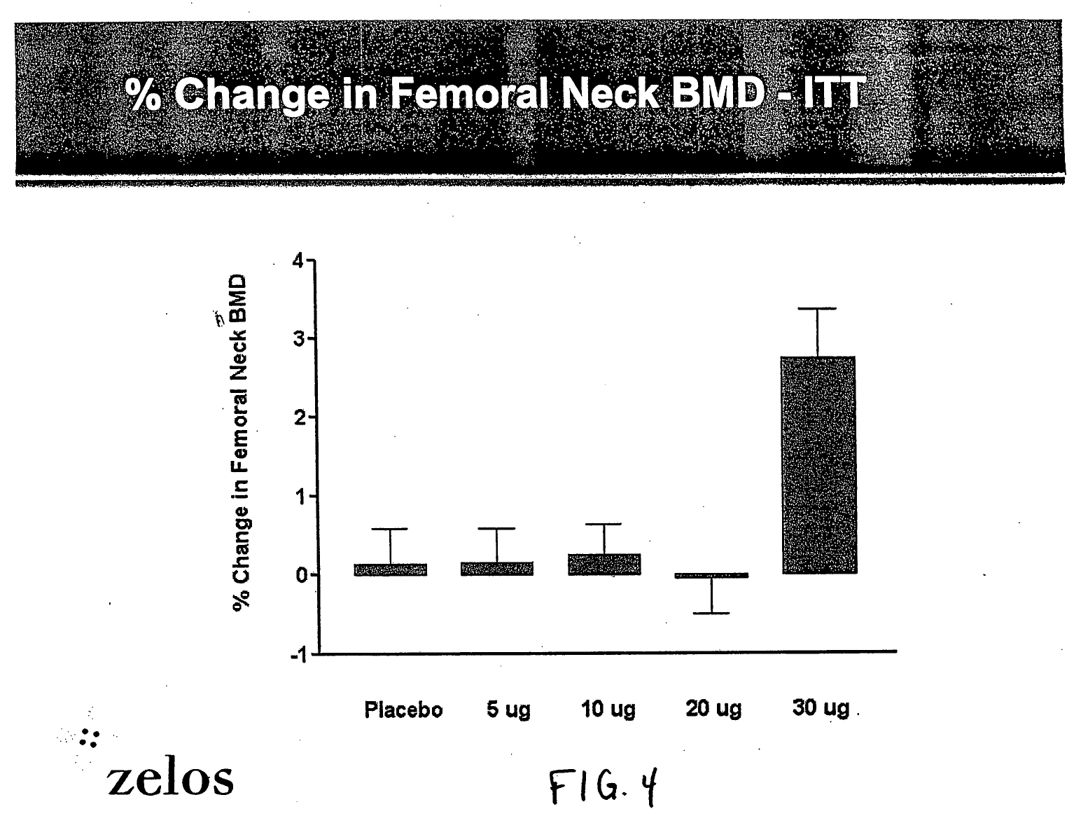

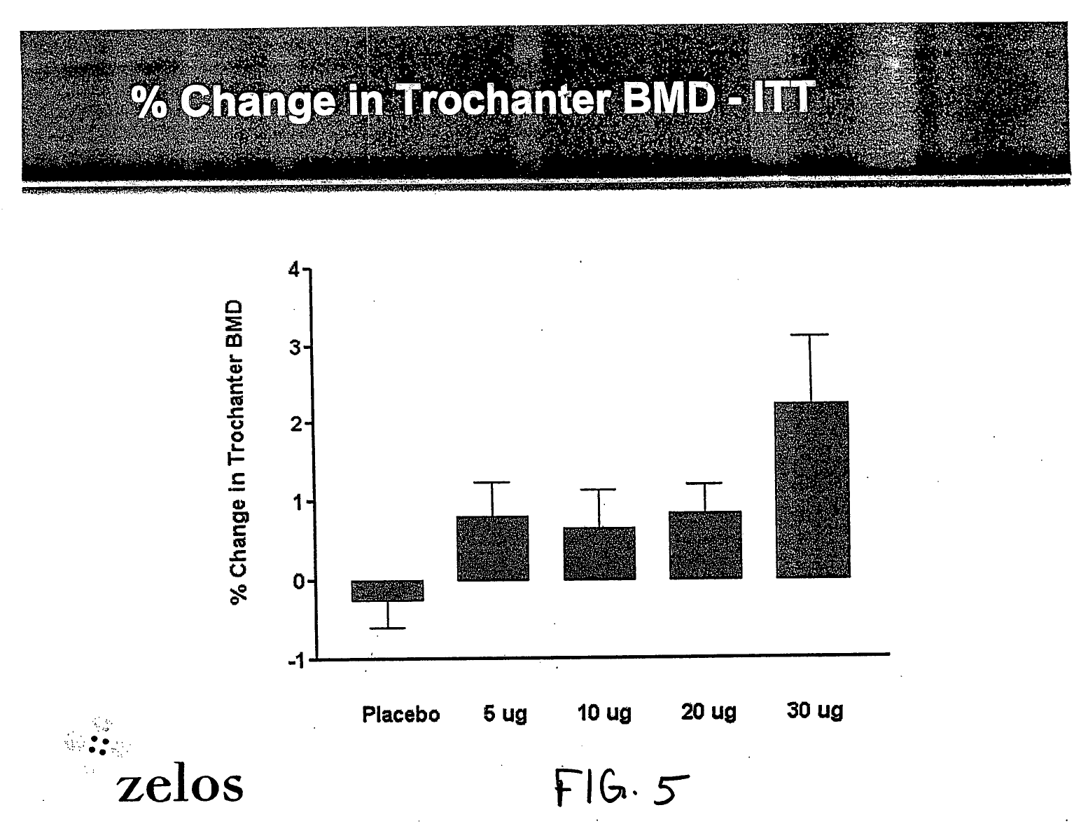

As shown in FIG. 1, administration of 5, 10, or 20 μg daily dosages of Ostabolin-C™ over a course of 15 weeks results in an increase in lumbar spine BMD. FIGS. 3, 4, and 5 demonstrate mild BMD increase in hip, femoral neck, and trochanter BMD following administration of Ostabolin-C™ for 15 weeks.

FIGS. 6 and 7 demonstrate that daily administration of 5, 10, and 20 μg of Ostabolin-C™ has an unexpectedly positive effect on cortical bone, specifically the wrist (the distal and mid-shaft radius). There were statistically significant effects at the mid-radius at daily dosages of 5, 10, 20 μg with no negative effect of bone resorption. Historically, PTH has been known to increase bone resorption, which leads to increased cortical porosity, and decreased BMD in radius cortical bone. Neer et al., 2001. As described in Neer, the administration of prior art Forteo® PTH 1-34 led to a decrease in BMD (increased cortical porosity) in the distal and mid-shaft radius as compared to placebo. In contrast, the dosages and formulation of the present invention, namely administration of 5, 10, and 20 μg Ostabolin-C™, actually increases cortical BMD in the distal and mid-shaft radius as compared to both placebo and to teriparatide, Forteo®. This is an unprecedented finding, demonstrating a statistically significant difference from placebo for 3 active doses (5, 10, 20 μg). FIGS. 8-13 demonstrate the effect which the PTHs of the present invention have on bone formation and bone resorption markers. The bone formation markers include P1NP, osteocalcin, and BSAP and the bone resorption markers include NTx and CTx. As compared to placebo, the bone formation markers have a greater % change when Ostabolin-C™ is administered at 10 and 20 μg.

The bone resorption markers in FIGS. 11-13 demonstrate that although there is some increase in bone resorption following the administration of Ostabolin-C™, this increase is less than that which follows administration of the prior art teriparatide, Forteo® PTH.

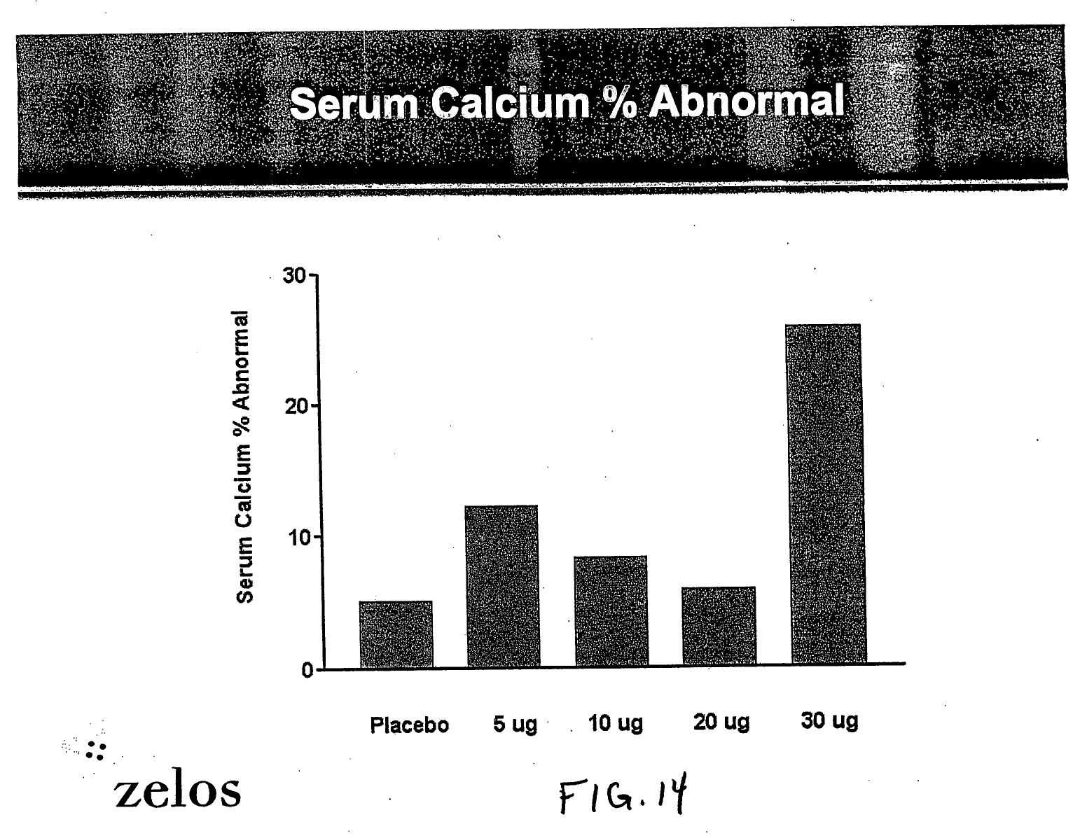

Daily dosages of 5, 10, and 20 μg Ostabolin-C™ has also been shown to have a much lower incidence of hypercalcemia as compared to PTHs known in the art. FIG. 14 demonstrates that there was no notable difference from placebo on the per cent of abnormal serum calcium for doses of Ostabolin-C™ up to and including 20 μg. In comparison, teriparatide, Forteo® is shown to have a much higher effect at similar doses. For patients receiving Forteo®, hypercalcemia was seen at least once in 11% of the 20 μg group subjects and in 28% of the 40 μg group subjects, as compared with 2% in the placebo group. Neer et al., 2001. In contrast, the administration of low doses of the PTH peptides of the present invention (5, 10, and 20 μg) resulted in no significant increase in the incidences of hypercalcemia as compared to placebo. Hypercalcemia was seen at least once in 5% of the placebo group and in the group being administered 20 μg doses, resulting in no net increase. This is in comparison to the 11% seen with Forteo® administered at 20 μg.

Accordingly, the above results demonstrate that administration of Ostabolin-C™ at 5, 10, and 20 μg daily dosages provides many advantages over the administration of Forteo® at 20 μg. The unexpected results include increased cortical BMD in the distal and mid-shaft radius as compared to placebo, less bone resorption than prior art PTH, and lower incidence and severity of hypercalcemia, while maintaining anabolic bone growth as measured by increased BMD at a variety of sites, including spine and hip.

### EXAMPLE 7

### Pre-Clinical Results—Effects of High Dose (30 μg) Ostabolin-C™

Administration of a daily dosage of 30 μg of Ostabolin-C™ has demonstrated an unprecedented ability to build bone at different sites, including the spine and hip, with early onset of effect in combination with only a mild hypercalcemia signal. This is an improvement over the prior art teriparatide, Forteo® 1-34 PTH.

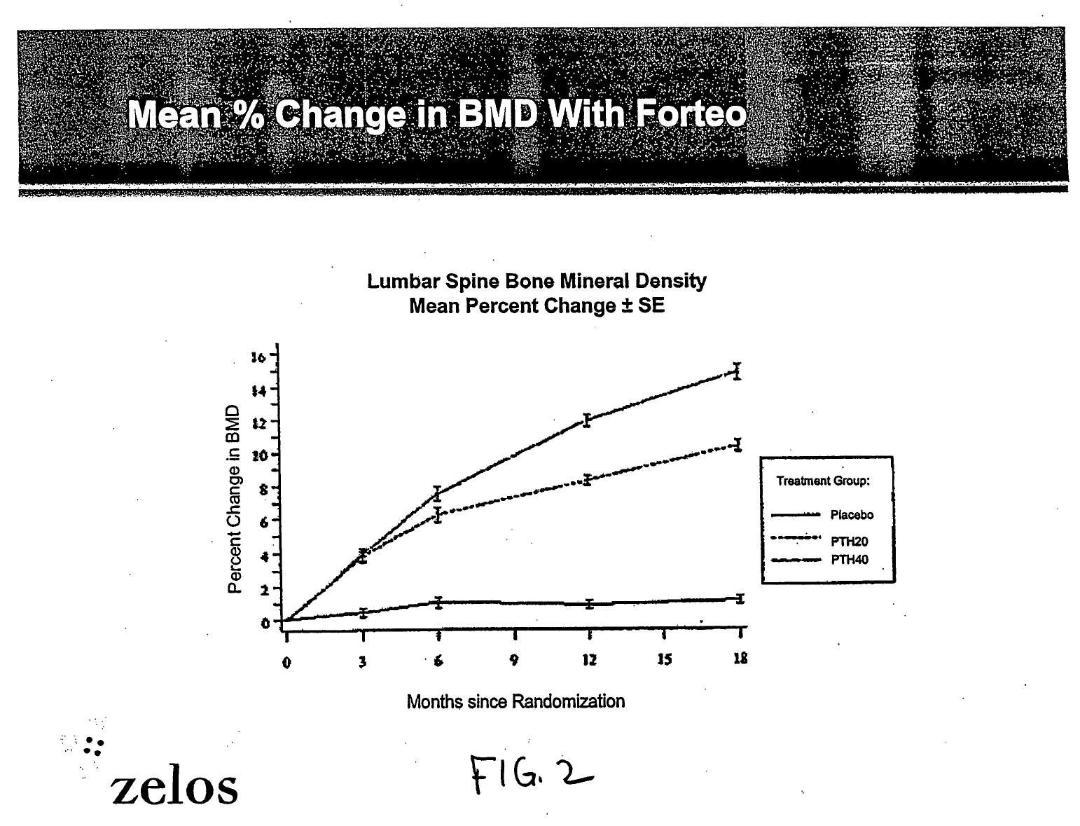

FIGS. 1 and 2 demonstrate that administration of 30 μg Ostabolin-C™ leads to an increase in BMD in the lumbar spine. FIG. 2 shows the increase in lumbar spine BMD with administration of 20 and 40 μg Forteo®.

FIGS. 3, 4, and 5 and the table below demonstrate that a daily dosage of 30 μg Ostabolin-C™ has a positive effect on bone formation at the hip, femoral neck, and trochanter. This is an unprecedented finding, demonstrating a statistically significant and clinically meaningful benefit at 30 μg at 15 weeks. The table below demonstrates the change in hip, femoral neck, and trochanter BMD, comparing the administration of teriparatide, Forteo® (20 μg) over a course of at least 12 months versus Ostabolin-C™ (30 μg) at 15 weeks. As shown below, for hip and trochanter, administration of 30 μg Ostabolin-C™ achieved results in 15 weeks similar to the results obtained with administration of Forteo over a course of at least 12 months. Regarding femoral neck, Ostabolin-C™ shows a much greater increase in BMD in a shorter period of time.

| | | TERIPARATIDE, |
|---|---|---|
| | Ostabolin-C | FORTEO ® 20 μg for |
| | 30 μg For 15 Weeks | at least 12 Months |
| Mean % Change In | 1.44 | 1.70 |
| Total Hip | | |
| Mean % Change In | 2.75 | 1.54 |
| Femoral Neck | | |
| Mean % Change In | 2.24 | 2.68 |
| Trochanter | | |

FIGS. 8-13 demonstrate the effect which the PTHs of the present invention have on bone formation and bone resorption markers. The bone formation markers include P1NP, osteocalcin, and BSAP and the bone resorption markers include NTx and CTx. As compared to placebo, the bone formation markers have a greater % change when Ostabolin-C™ is administered at 30 μg. There is a robust effect in the increase in the bone formation markers when Ostabolin-C™ is administered at 30 μg. The bone resorption markers in FIGS. 11-13 demonstrate that although there is some increase in bone resorption following the administration of Ostabolin-C™, this increase is less than that which follows administration of the prior art teriparatide, Forteo® PTH.

Accordingly, the above results demonstrate that administration of Ostabolin-C™ at 30 μg daily dosages provides many advantages over the administration of rhPTH 1-34 teriparatide, Forteo® at 20 and 40 μg. The unexpected results include increased BMD in the spine and hip, with less bone resorption and lower incidences of hypercalcemia than prior art PTH.

### EXAMPLE 8

### Pharmacokinetic (PK) Evaluation of Ostabolin-C

The objective of this portion of the study was to evaluate the pharmacokinetics of Ostabolin-C under steady state conditions when given subcutaneously (sc) once a day to post-menopausal female subjects with low bone mineral density.

This study was a Phase II, multicenter, randomized, double-blind, placebo-controlled, parallel group dose-finding study in post-menopausal female subjects. After Screening procedures and a 2-week placebo run-in phase, subjects were to be dosed once a day for 16 weeks with either Placebo or Ostabolin-C (5, 10, 20 or 30 μg). A subset of subjects from all treatment groups had blood collected for measurements of Ostabolin-C in order to determine PK parameters and compare them to prior studies.

The full study duration of the study was 22 weeks, which included a 6-week screening period involving a 2-week placebo run-in and then 16 weeks of treatment. The subset of subjects for this component of the study was treated the same as all other subjects with the exception of the additional blood collections at baseline and Week 12.

For this component of the study, additional blood samples were collected in lithium-heparin containing tubes at 15 minutes, 30 minutes, 1 hr, 2 hr, 4 hr, 6 hr and 24 hr post-dose at the Baseline and Week 12 clinic visits. Subjects were allowed to go home and return between the 6 and 24 hour sampling times. Plasma samples were frozen and shipped to the following laboratory for analysis of Ostabolin-C content using a validated ELISA assay:
Covance Laboratories Limited
Otley Road, Harrogate
North Yorkshire
England HG3 1PY

Elisa

Analyte: Ostabolin-C

Quantitation limit: 10.0 pg/mL
Sample analysis calibration curve range: 5.0 pg/mL to 800 pg/mL
Between-run CV of LoQC (50.00 pg/mL) (Precision): 7.9%
Between-run % nominal of LoQC (50.00 pg/mL) (Accuracy): 115.8%

Data Handling and PK Analyses

The raw data as provided by Covance are presented in the Table entitled “Raw Data” below.

For values below the level of assay detection (i.e., 10 pg/ml) the values were set to zero to estimate the PK parameters.

All of the values from the Placebo subject except one (Baseline 2 hour time-point) were below the assay level of detection (i.e., 10 pg/ml). With very limited exceptions, all values from Placebo subjects in prior trials have also been below the levels of detection. Thus, this one value was considered to be a laboratory error and PK parameters for this placebo subject were not calculated.

No samples were obtained for Pre-dose at either Baseline or Week 12. In all prior studies, pre-treatment values have been below the levels of detection and 24-hour time points at doses of 40 ug and below have been below the levels of detection. Thus, no observable values were anticipated and for calculating PK the pre-treatment values for Baseline visit were set to zero.

For pre-dose at Week 12, it was also anticipated that the values would be below the levels of detection based on prior studies and that the 24-hour post dose value would verify this. Only two 24-hour time-point values were above the level of detection; i.e. 24 hours post dosing at Baseline for Subject 030-003 and 24 hours post dosing at Week 12 for Subject 030-0004. The values of both of these 24-hour time-points were marginally above the assay level of detection. Also, both of these subjects had values below the levels of detection for both the 4 hour and 6 hour time-points after dosing on the prior day. Thus, these values are most likely artifacts and not real values. No valid 24 hour sample for subject 032-0001 at Week 12 was obtained. However, the 6 hour time point after dosing at Week 12 was below the levels of detection, and thus the value for 24-hours was assumed to be also below the level of detection to estimate the AUC(0-24) value. Thus, for calculating PK parameters, the Pre-dose values for Week 12 were also set to zero.

The pharmacokinetic parameters that were estimated at Baseline and Week 12 are as follows:
The area under the drug concentration-time curve from time zero to time 4 hours (AUC₍₀₋₄₎)
The area under the drug concentration-time curve from time zero to time 24 hours (AUC₍₀₋₂₄₎)
The maximum observed drug concentration (Cₘₐₓ)
The time of the maximum drug concentration (Tₘₐₓ)

Since so few subjects were included in this subset of subjects and the time points used for collections were limited, additional PK parameters were not calculated.

AUC values were estimated by a simple summation of trapezoidal areas from each time period. Data from each dose group were summarized using simple statistics on an Excel® spreadsheet; i.e., average (AVG) and Standard Deviation (STD). It should be noted that particularly with the lower doses and associated low blood levels and at late time points, those values just above versus just below the assay limits of detection can have a disproportionate impact to AUC calculations. This adds to that variability of the calculated numbers.

PK Values

The following table summarizes the estimated PK parameters.

BASELINE PK PARAMETERS

| | | | | | |
|---|---|---|---|---|---|
| Site/sub | Date | Cmax | Tmax | AUC(0-24) | AUC(0-4) |

Dose Group = 5 ug

| | | | | | |
|---|---|---|---|---|---|
| 006-0063 | 18 Oct. 2005 | 20.7 | 0.25 | 11.71 | 11.71 |
| 030-0005 | 19 Oct. 2005 | 27.75 | 0.25 | 16.25 | 16.25 |
| 038-0010 | 11 Jan. 2006 | 33.15 | 1.00 | 99.67 | 88.35 |
| | AVG | 27.20 | 0.50 | 42.54 | 38.77 |
| | STD | 6.24 | 0.43 | 49.52 | 43.00 |

Dose Group = 10 ug

| | | | | | |
|---|---|---|---|---|---|
| 030-0003 | 10 Aug. 2005 | 30.6 | 0.50 | 65.98 | 65.98 |
| 032-0001 | 13 Jun. 2005 | 57.54 | 0.25 | 82.31 | 82.31 |
| 038-0001 | 8 Nov. 2005 | 46.59 | 0.25 | 74.38 | 74.38 |
| | AVG | 44.91 | 0.33 | 74.22 | 74.22 |
| | STD | 13.55 | 0.14 | 8.17 | 8.17 |

Dose Group = 20 ug

| | | | | | |
|---|---|---|---|---|---|
| 006-0144 | 16 Dec. 2005 | 63.93 | 0.50 | 104.11 | 104.11 |

Dose Group = 30 ug

| | | | | | |
|---|---|---|---|---|---|
| 006-0141 | 13 Dec. 2005 | 114.54 | 0.50 | 469.78 | 337.16 |
| 030-0004 | 16 Nov. 2005 | 233.33 | 0.50 | 370.47 | 344.39 |
| | AVG | 173.94 | 0.50 | 420.12 | 340.77 |

Since there are so few subjects that actually participated in this part of the study and since the values for Cmax, Tmax, and AUC appeared to be very similar for both Baseline and Week 12, the values for all times were averaged to obtain another estimate of these parameters; see table below. Similar PK values for Baseline and Day 7 when steady state kinetics should have been have reached equilibrium have been seen in two previous Phase 1 studies involving this dose range.

WEEK 12 PK PARAMETERS

| | | | | | |
|---|---|---|---|---|---|
| Site/sub | Date | Cmax | Tmax | AUC(0-24) | AUC(0-4) |

Dose Group = 5 ug

| | | | | | |
|---|---|---|---|---|---|
| 006-0063 | 17 Jan. 2006 | 21.04 | 0.25 | 9.10 | 9.10 |
| 030-0005 | 11 Jan. 2006 | 37.84 | 0.25 | 28.83 | 28.83 |
| 038-0010 | 5 Apr. 2006 | 40.38 | 0.25 | 64.83 | 64.83 |
| | AVG | 33.09 | 0.25 | 34.25 | 34.25 |
| | STD | 10.51 | 0.00 | 28.26 | 28.26 |

Dose Group = 10 ug

| | | | | | |
|---|---|---|---|---|---|
| 030-0003 | 2 Nov. 2005 | 41.98 | 0.25 | 164.36 | 68.69 |
| 032-0001 | 14 Sep. 2005 | 33.33 | 0.25 | 54.29* | 54.29 |
| 038-0001 | 24 Jan. 2006 | 69.33 | 0.50 | 110.80 | 110.80 |
| | AVG | 48.21 | 0.33 | 109.82 | 77.93 |
| | STD | 18.79 | 0.14 | 55.04 | 29.37 |

Dose Group = 20 ug

| | | | | | |
|---|---|---|---|---|---|
| 006-0144 | 13 Mar. 2006 | 76.25 | 0.25 | 153.89 | 142.45 |

Dose Group = 30 ug

| | | | | | |
|---|---|---|---|---|---|
| 006-0141 | 15 Mar. 2006 | 46.75 | 0.25 | 214.03 | 70.73 |
| 030-0004 | 8 Feb. 2006 | 87.1 | 0.25 | 128.37 | 128.37 |
| | AVG | 66.93 | 0.25 | 171.20 | 99.55 |

*Note -
no valid 24 hour sample for subject 032-0001 was obtained but since the 6 hour time point was below the levels of detection, the value for 24-hours was assumed to be also below the level of detection to estimate the AUC(0-24) value.

ESTIMATED PK PARAMETERS - AVERAGE
OF BASELINE AND WEEK 12 DATA

| | | | Cmax | Tmax | AUC(0-24) | AUC(0-4) |
|---|---|---|---|---|---|---|
| Dose | [N]* | | pg/ml | Hours | pg · hr/ml | pg · hr/ml |
| 5 | 3 | Mean | 30.14 | 0.38 | 38.40 | 36.51 |
| | | Std | 8.38 | 0.31 | 36.35 | 32.63 |
| 10 | 3 | Mean | 46.56 | 0.33 | 92.02 | 76.07 |
| | | Std | 14.76 | 0.13 | 40.23 | 19.38 |
| 20 | 1 | Mean | 70.09 | 0.38 | 129.00 | 123.28 |
| | | Std | 8.71 | 0.18 | 35.20 | 27.11 |
| 30 | 2 | Mean | 120.43 | 0.38 | 295.66 | 220.16 |
| | | Std | 80.25 | 0.14 | 153.37 | 141.28 |

*Note:
N = number of subjects; data from both Baseline and Week 12 combined, each subject had two values for each parameter.

Since Tₘₐₓ seems to be dose independent in this study as well as in previous studies, the Tₘₐₓ from all doses in this study determined at Baseline and Week 12 were averaged to obtain an overall estimated value of 0.34 hours with a STD of 0.21 hrs.

Discussion

The very limited numbers of subjects involved in this study limit the statistical confidence in the conclusions drawn from the data in this study. However, the data are basically consistent with prior studies.

As seen in prior studies, there was no evidence of accumulation. The PK parameters after 12 weeks of dosing were very similar to those on Day 1 at Baseline.

The Tmax was independent of dose and the overall average form all doses and times was 0.34 hours (srd=0.21 hrs).

The Cmax and the AUC values increased with dose. There is a rough dose relationship with Cmax and AUVC values in the averaged data.

Raw Data

Analysis of Human Plasma and Serum Samples (Phase II) for the Detection of
Ostabolin-C and anti-Ostabolin-C antibodies Using ELISA.
PK analysis Sample Data

| Acc-ctn | Patient | Invest. | | | Date of | | | |
|---|---|---|---|---|---|---|---|---|
| Number | Number | Number | Visit Description | | Collection | Result | Units | Comment |
| H270478-10 | 006-0063 | 006 | 15 | MIN/PLASMA | 18 Oct. 2005 | 20.70 | pg/mL | |
| H270478-13 | 006-0063 | 006 | 30 | MIN/PLASMA | 18 Oct. 2005 | 17.42 | pg/mL | |
| H270478-16 | 006-0063 | 006 | 1 | HR/PLASMA | 18 Oct. 2005 | <10.00 | pg/mL | |
| H270478-19 | 006-0063 | 006 | 2 | HR/PLASMA | 18 Oct. 2005 | <10.00 | pg/mL | |
| H270478-22 | 006-0063 | 006 | 4 | HR/PLASMA | 18 Oct. 2005 | <10.00 | pg/mL | |
| H270478-25 | 006-0063 | 006 | 6 | HR/PLASMA | 18 Oct. 2005 | <10.00 | pg/mL | |
| H270478-28 | 006-0063 | 006 | 24 | HR/PLASMA | 19 Oct. 2005 | <10.00 | pg/mL | |
| H197596-10 | 006-0141 | 006 | 15 | MIN/PLASMA | 13 Dec. 2005 | 98.21 | pg/mL | |
| H197596-13 | 006-0141 | 006 | 30 | MIN/PLASMA | 13 Dec. 2005 | 114.54 | pg/mL | |
| H197596-16 | 006-0141 | 006 | 1 | HR/PLASMA | 13 Dec. 2005 | 67.72 | pg/mL | |
| H197596-19 | 006-0141 | 006 | 2 | HR/PLASMA | 13 Dec. 2005 | 134.03 | pg/mL | |
| H197596-22 | 006-0141 | 006 | 4 | HR/PLASMA | 13 Dec. 2005 | 17.82 | pg/mL | |
| H197596-25 | 006-0141 | 006 | 6 | HR/PLASMA | 13 Dec. 2005 | 11.48 | pg/mL | |
| H197596-28 | 006-0141 | 006 | 24 | HR/PLASMA | 14 Dec. 2005 | <10.00 | pg/mL | |
| I007538-10 | 006-0144 | 006 | 15 | MIN/PLASMA | 16 Dec. 2005 | 63.65 | pg/mL | |
| I007538-13 | 006-0144 | 006 | 30 | MIN/PLASMA | 16 Dec. 2005 | 63.93 | pg/mL | |
| I007538-16 | 006-0144 | 006 | 1 | HR/PLASMA | 16 Dec. 2005 | 46.85 | pg/mL | |
| I007538-19 | 006-0144 | 006 | 2 | HR/PLASMA | 16 Dec. 2005 | 19.39 | pg/mL | |
| I007538-22 | 006-0144 | 006 | 4 | HR/PLASMA | 16 Dec. 2005 | <10.00 | pg/mL | |
| I007538-25 | 006-0144 | 006 | 6 | HR/PLASMA | 16 Dec. 2005 | <10.00 | pg/mL | |
| I007538-28 | 006-0144 | 006 | 24 | HR/PLASMA | 17 Dec. 2005 | <10.00 | pg/mL | |
| E504107-10 | 030-0003 | 030 | 15 | MIN/PLASMA | 10 Aug. 2005 | 28.88 | pg/mL | |
| E504107-13 | 030-0003 | 030 | 30 | MIN/PLASMA | 10 Aug. 2005 | 30.60 | pg/mL | |
| E504107-16 | 030-0003 | 030 | 1 | HR/PLASMA | 10 Aug. 2005 | 23.71 | pg/mL | |
| E504107-19 | 030-0003 | 030 | 2 | HR/PLASMA | 10 Aug. 2005 | 19.67 | pg/mL | |
| E504107-22 | 030-0003 | 030 | 4 | HR/PLASMA | 10 Aug. 2005 | <10.00 | pg/mL | |
| E504107-25 | 030-0003 | 030 | 6 | HR/PLASMA | 10 Aug. 2005 | <10.00 | pg/mL | |
| E504107-28 | 030-0003 | 030 | 24 | HR/PLASMA | 11 Aug. 2005 | <10.00 | pg/mL | |
| G491569-10 | 030-0004 | 030 | 15 | MIN/PLASMA | 16 Nov. 2005 | 189.91 | pg/mL | |
| G491569-13 | 030-0004 | 030 | 30 | MIN/PLASMA | 16 Nov. 2005 | 233.33 | pg/mL | |
| G491569-16 | 030-0004 | 030 | 1 | HR/PLASMA | 16 Nov. 2005 | 130.48 | pg/mL | |
| G491569-19 | 030-0004 | 030 | 2 | HR/PLASMA | 16 Nov. 2005 | 56.98 | pg/mL | |
| G491569-22 | 030-0004 | 030 | 4 | HR/PLASMA | 16 Nov. 2005 | 26.08 | pg/mL | |
| G491569-25 | 030-0004 | 030 | 6 | HR/PLASMA | 16 Nov. 2005 | <10.00 | pg/mL | |
| G491569-28 | 030-0004 | 030 | 24 | HR/PLASMA | 17 Nov. 2005 | <10.00 | pg/mL | |
| G491576-10 | 030-0005 | 030 | 15 | MIN/PLASMA | 19 Oct. 2005 | 27.75 | pg/mL | |
| G491576-13 | 030-0005 | 030 | 30 | MIN/PLASMA | 19 Oct. 2005 | 24.82 | pg/mL | |
| G491576-16 | 030-0005 | 030 | 1 | HR/PLASMA | 19 Oct. 2005 | <10.00 | pg/mL | |
| G491576-19 | 030-0005 | 030 | 2 | HR/PLASMA | 19 Oct. 2005 | <10.00 | pg/mL | |
| G491576-22 | 030-0005 | 030 | 4 | HR/PLASMA | 19 Oct. 2005 | <10.00 | pg/mL | |
| G491576-25 | 030-0005 | 030 | 6 | HR/PLASMA | 19 Oct. 2005 | <10.00 | pg/mL | |
| G491576-28 | 030-0005 | 030 | 24 | HR/PLASMA | 20 Oct. 2005 | <10.00 | pg/mL | |
| E504149-10 | 032-0001 | 032 | 15 | MIN/PLASMA | 13 Jun. 2005 | 57.54 | pg/mL | |
| E504149-13 | 032-0001 | 032 | 30 | MIN/PLASMA | 13 Jun. 2005 | 52.84 | pg/mL | |
| E504149-16 | 032-0001 | 032 | 1 | HR/PLASMA | 13 Jun. 2005 | 35.43 | pg/mL | |
| E504149-19 | 032-0001 | 032 | 2 | HR/PLASMA | 13 Jun. 2005 | 14.36 | pg/mL | |
| E504149-22 | 032-0001 | 032 | 4 | HR/PLASMA | 13 Jun. 2005 | <10.00 | pg/mL | |
| E504149-25 | 032-0001 | 032 | 6 | HR/PLASMA | 13 Jun. 2005 | <10.00 | pg/mL | |
| E504149-28 | 032-0001 | 032 | 24 | HR/PLASMA | 14 Jun. 2005 | 18.82 | pg/mL | |
| I007528-10 | 006-0141 | 006 | 15 | MIN/PLASMA | 13 Mar. 2006 | 76.25 | pg/mL | |
| I007528-13 | 006-0141 | 006 | 30 | MIN/PLASMA | 13 Mar. 2006 | 64.16 | pg/mL | |
| I007528-16 | 006-0141 | 006 | 1 | HR/PLASMA | 13 Mar. 2006 | 42.54 | pg/mL | |
| I007528-19 | 006-0141 | 006 | 2 | HR/PLASMA | 13 Mar. 2006 | 37.32 | pg/mL | |
| I007528-22 | 006-0141 | 006 | 4 | HR/PLASMA | 13 Mar. 2006 | 11.44 | pg/mL | |
| I007528-25 | 006-0141 | 006 | 6 | HR/PLASMA | 13 Mar. 2006 | <10.00 | pg/mL | |
| I007528-28 | 006-0141 | 006 | 24 | HR/PLASMA | 14 Mar. 2006 | <10.00 | pg/mL | |
| I007529-10 | 006-0144 | 006 | 15 | MIN/PLASMA | 15 Mar. 2006 | 46.75 | pg/mL | |
| I007529-13 | 006-0144 | 006 | 30 | MIN/PLASMA | 15 Mar. 2006 | 40.04 | pg/mL | |
| I007529-16 | 006-0144 | 006 | 1 | HR/PLASMA | 15 Mar. 2006 | 24.34 | pg/mL | |
| I007529-19 | 006-0144 | 006 | 2 | HR/PLASMA | 15 Mar. 2006 | 17.18 | pg/mL | |
| I007529-22 | 006-0144 | 006 | 4 | HR/PLASMA | 15 Mar. 2006 | <10.00 | pg/mL | |
| I007529-25 | 006-0144 | 006 | 6 | HR/PLASMA | 15 Mar. 2006 | 14.33 | pg/mL | |
| I007529-28 | 006-0144 | 006 | 24 | HR/PLASMA | 16 Mar. 2006 | <10.00 | pg/mL | |
| E504106-10 | 030-0004 | 030 | 15 | MIN/PLASMA | 8 Feb. 2006 | 87.10 | pg/mL | |
| E504106-13 | 030-0004 | 030 | 30 | MIN/PLASMA | 8 Feb. 2006 | 80.72 | pg/mL | |
| E504106-16 | 030-0004 | 030 | 1 | HR/PLASMA | 8 Feb. 2006 | 61.92 | pg/mL | |
| E504106-19 | 030-0004 | 030 | 2 | HR/PLASMA | 8 Feb. 2006 | 19.92 | pg/mL | |
| E504106-22 | 030-0004 | 030 | 4 | HR/PLASMA | 8 Feb. 2006 | <10.00 | pg/mL | |
| E504106-25 | 030-0004 | 030 | 6 | HR/PLASMA | 8 Feb. 2006 | <10.00 | pg/mL | |
| E504106-28 | 030-0004 | 030 | 24 | HR/PLASMA | 9 Feb. 2006 | 14.12 | pg/mL | |
| H200077-10 | 038-0001 | 038 | 15 | MIN/PLASMA | 8 Nov. 2005 | 46.59 | pg/mL | |
| H200077-13 | 038-0001 | 038 | 30 | MIN/PLASMA | 8 Nov. 2005 | 28.10 | pg/mL | |
| H200077-16 | 038-0001 | 038 | 1 | HR/PLASMA | 8 Nov. 2005 | 34.81 | pg/mL | |
| H200077-19 | 038-0001 | 038 | 2 | HR/PLASMA | 8 Nov. 2005 | 17.39 | pg/mL | |
| H200077-22 | 038-0001 | 038 | 4 | HR/PLASMA | 8 Nov. 2005 | <10.00 | pg/mL | |
| H200077-25 | 038-0001 | 038 | 6 | HR/PLASMA | 8 Nov. 2005 | <10.00 | pg/mL | |
| H200077-28 | 038-0001 | 038 | 24 | HR/PLASMA | 9 Nov. 2005 | <10.00 | pg/mL | |
| I495139-10 | 038-0001 | 038 | 15 | MIN/PLASMA | 24 Jan. 2006 | 62.75 | pg/mL | |
| I495139-13 | 038-0001 | 038 | 30 | MIN/PLASMA | 24 Jan. 2006 | 69.33 | pg/mL | |
| I495139-16 | 038-0001 | 038 | 1 | HR/PLASMA | 24 Jan. 2006 | 48.05 | pg/mL | |
| I495139-19 | 038-0001 | 038 | 2 | HR/PLASMA | 24 Jan. 2006 | 22.05 | pg/mL | |
| I495139-22 | 038-0001 | 038 | 4 | HR/PLASMA | 24 Jan. 2006 | <10.00 | pg/mL | |
| I495139-25 | 038-0001 | 038 | 6 | HR/PLASMA | 24 Jan. 2006 | <10.00 | pg/mL | |
| I495139-28 | 038-0001 | 038 | 24 | HR/PLASMA | 25 Jan. 2006 | <10.00 | pg/mL | |
| H714066-10 | 038-0008 | 038 | 15 | MIN/PLASMA | 10 Jan. 2006 | <10.00 | pg/mL | |
| H714066-13 | 038-0008 | 038 | 30 | MIN/PLASMA | 10 Jan. 2006 | <10.00 | pg/mL | |
| H714066-16 | 038-0008 | 038 | 1 | HR/PLASMA | 10 Jan. 2006 | <10.00 | pg/mL | |
| H714066-19 | 038-0008 | 038 | 2 | HR/PLASMA | 10 Jan. 2006 | 46.29 | pg/mL | |
| H714066-22 | 038-0008 | 038 | 4 | HR/PLASMA | 10 Jan. 2006 | <10.00 | pg/mL | |
| H714066-25 | 038-0008 | 038 | 6 | HR/PLASMA | 10 Jan. 2006 | <10.00 | pg/mL | |
| H714065-10 | 038-0008 | 038 | 15 | MIN/PLASMA | 4 Apr. 2006 | <10.00 | pg/mL | |
| H714065-13 | 038-0008 | 038 | 30 | MIN/PLASMA | 4 Apr. 2006 | <10.00 | pg/mL | |
| H714065-16 | 038-0008 | 038 | 1 | HR/PLASMA | 4 Apr. 2006 | <10.00 | pg/mL | |
| H714065-19 | 038-0008 | 038 | 2 | HR/PLASMA | 4 Apr. 2006 | <10.00 | pg/mL | |
| H714065-22 | 038-0008 | 038 | 4 | HR/PLASMA | 4 Apr. 2006 | <10.00 | pg/mL | |
| H714065-25 | 038-0008 | 038 | 6 | HR/PLASMA | 4 Apr. 2006 | <10.00 | pg/mL | |
| H714065-28 | 038-0008 | 038 | 24 | HR/PLASMA | 5 Apr. 2006 | <10.00 | pg/mL | |
| H200079-10 | 038-0010 | 038 | 15 | MIN/PLASMA | 11 Jan. 2006 | 26.94 | pg/mL | |
| H200079-13 | 038-0010 | 038 | 30 | MIN/PLASMA | 11 Jan. 2006 | 32.06 | pg/mL | |
| H200079-16 | 038-0010 | 038 | 1 | HR/PLASMA | 11 Jan. 2006 | 33.15 | pg/mL | |
| H200079-19 | 038-0010 | 038 | 2 | HR/PLASMA | 11 Jan. 2006 | 22.27 | pg/mL | |
| H200079-22 | 038-0010 | 038 | 4 | HR/PLASMA | 11 Jan. 2006 | 11.32 | pg/mL | |
| H200079-25 | 038-0010 | 038 | 6 | HR/PLASMA | 11 Jan. 2006 | <10.00 | pg/mL | |
| H200079-28 | 038-0010 | 038 | 24 | HR/PLASMA | 12 Jan. 2006 | <10.00 | pg/mL | |
| I495146-10 | 038-0010 | 038 | 15 | MIN/PLASMA | 5 Apr. 2006 | 40.38 | pg/mL | |
| I495146-13 | 038-0010 | 038 | 30 | MIN/PLASMA | 5 Apr. 2006 | 31.77 | pg/mL | |
| I495146-16 | 038-0010 | 038 | 1 | HR/PLASMA | 5 Apr. 2006 | 27.05 | pg/mL | |
| I495146-19 | 038-0010 | 038 | 2 | HR/PLASMA | 5 Apr. 2006 | 15.02 | pg/mL | |
| I495146-22 | 038-0010 | 038 | 4 | HR/PLASMA | 5 Apr. 2006 | <10.00 | pg/mL | |
| I495146-25 | 038-0010 | 038 | 6 | HR/PLASMA | 5 Apr. 2006 | <10.00 | pg/mL | |
| I495146-28 | 038-0010 | 038 | 24 | HR/PLASMA | 6 Apr. 2006 | <10.00 | pg/mL | |
| I007537-10 | 006-0063 | 006 | 15 | MIN/PLASMA | 17 Jan. 2006 | 21.04 | pg/mL | |
| I007537-13 | 006-0063 | 006 | 30 | MIN/PLASMA | 17 Jan. 2006 | 10.24 | pg/mL | |
| I007537-16 | 006-0063 | 006 | 1 | HR/PLASMA | 17 Jan. 2006 | <10.00 | pg/mL | |
| I007537-19 | 006-0063 | 006 | 2 | HR/PLASMA | 17 Jan. 2006 | <10.00 | pg/mL | |
| I007537-22 | 006-0063 | 006 | 4 | HR/PLASMA | 17 Jan. 2006 | <10.00 | pg/mL | |
| I007537-25 | 006-0063 | 006 | 6 | HR/PLASMA | 17 Jan. 2006 | <10.00 | pg/mL | |
| I007537-28 | 006-0063 | 006 | 24 | HR/PLASMA | 18 Jan. 2006 | <10.00 | pg/mL | |
| G491578-10 | 030-0003 | 030 | 15 | MIN/PLASMA | 2 Nov. 2005 | 41.98 | pg/mL | |
| G491578-13 | 030-0003 | 030 | 30 | MIN/PLASMA | 2 Nov. 2005 | 36.71 | pg/mL | |
| G491578-16 | 030-0003 | 030 | 1 | HR/PLASMA | 2 Nov. 2005 | 27.64 | pg/mL | |
| G491578-19 | 030-0003 | 030 | 2 | HR/PLASMA | 2 Nov. 2005 | 15.80 | pg/mL | |
| G491578-22 | 030-0003 | 030 | 4 | HR/PLASMA | 2 Nov. 2005 | <10.00 | pg/mL | |
| G491578-25 | 030-0003 | 030 | 6 | HR/PLASMA | 2 Nov. 2005 | <10.00 | pg/mL | |
| G491578-28 | 030-0003 | 030 | 24 | HR/PLASMA | 3 Nov. 2005 | 10.63 | pg/mL | |
| G491579-10 | 030-0005 | 030 | 15 | MIN/PLASMA | 11 Jan. 2006 | 37.84 | pg/mL | |
| G491579-13 | 030-0005 | 030 | 30 | MIN/PLASMA | 11 Jan. 2006 | 22.71 | pg/mL | |
| G491579-16 | 030-0005 | 030 | 1 | HR/PLASMA | 11 Jan. 2006 | 14.47 | pg/mL | |
| G491579-19 | 030-0005 | 030 | 2 | HR/PLASMA | 11 Jan. 2006 | <10.00 | pg/mL | |
| G491579-22 | 030-0005 | 030 | 4 | HR/PLASMA | 11 Jan. 2006 | <10.00 | pg/mL | |
| G491579-25 | 030-0005 | 030 | 6 | HR/PLASMA | 11 Jan. 2006 | <10.00 | pg/mL | |
| G491579-28 | 030-0005 | 030 | 24 | HR/PLASMA | 12 Jan. 2006 | <10.00 | pg/mL | |
| F841148-10 | 032-0001 | 032 | 15 | MIN/PLASMA | 14 Sep. 2005 | 33.33 | pg/mL | |
| F841148-13 | 032-0001 | 032 | 30 | MIN/PLASMA | 14 Sep. 2005 | 32.62 | pg/mL | |
| F841148-16 | 032-0001 | 032 | 1 | HR/PLASMA | 14 Sep. 2005 | 24.12 | pg/mL | |
| F841148-19 | 032-0001 | 032 | 2 | HR/PLASMA | 14 Sep. 2005 | 10.42 | pg/mL | |
| F841148-22 | 032-0001 | 032 | 4 | HR/PLASMA | 14 Sep. 2005 | <10.00 | pg/mL | |
| F841148-25 | 032-0001 | 032 | 6 | HR/PLASMA | 14 Sep. 2005 | <10.00 | pg/mL | |

### EXAMPLE 9

### Treatment of Renal Osteodystrophy

End stage renal disease is invariably associated with bone disease, known as renal osteodystrophy (ROD) (for account of pathogenesis see Primer on Metabolic Bone Diseases and Disorders of Mineral Metabolism Chapter 74). ROD may exist in a high turnover form characterized by high circulating levels of PTH (secondary hyperparathyroidism) and overactive bone tissue. This condition is frequently associated with bone pain, muscle weakness, extraskeletal calcification and deformities and growth retardation in children. Reduction in PTH levels is considered necessary to treat these problems. The low turnover form of the disease, also known as adynamic bone disease, is characterized by normal or low circulating levels of PTH and is increasing in incidence due to the increasing use of therapies to effectively control secondary hyperparathyroidism such as Vitamin D sterols, calcium based phosphate binding agents and calcimimetic drugs. Histologically the bone surfaces are quiescent with little or no osteoblast cellular activity. Clinical consequences of this histological state include increased risk of fractures and growth retardation in prepubertal children.

Adynamic bone disease is currently difficult to treat. The use of parathyroid hormone is contraindicated since reducing parathyroid hormone levels is one of the important goals of the therapies that lead to adynamic disease. Hypercalcemia is a frequent complication of current therapeutic strategies and this would be exacerbated by the use of exogenous PTH. Restoration of normal levels of bone formation activity is therefore difficult to achieve in this setting and there is an unmet need for effective therapy. Agonists of the PTH receptor, exemplified by cyclized or linear PTH (1-31) analogs but also including other cyclic and linear analogs of smaller size and analogs of PTHrP have been shown to increase bone formation but do not have the propensity to stimulate bone resorption that is seen with other PTH fragments and with the naturally occurring hormone. PTH receptor agonists of this type may be able to stimulate osteoblastic function and bone formation and thus effectively treat adynamic bone disease without exacerbation of the risk of hypercalcemia. The use of low doses of these agents may be particularly effective in prevention and treatment of adynamic bone disease to provide restoration of normal osteoblast activity with minimal bone resorption stimulating activity. Specific treatment scenarios in which PTH receptor agonists of this type are used in combination with calcimimetic drugs, Vitamin D sterols or other agents known to increase the occurrence and/or severity of adynamic bone disease to prevent this occurrence or exacerbation could be created.

PTH receptor agonists could be used in dialysis patients at increased risk of developing adynamic bone disease to prevent the occurrence of adynamic bone disease. PTH receptor agonists of the type described above could also be used to treat patients with osteoporosis and renal disease who have a particularly high risk of fracture due to adynamic bone disease.

### EXAMPLE 10

### Rat Oncogenicity Study

Prior art PTHs cause osteosarcomas in animals if administered over a course of two years. The PTH peptides of the present invention, including Ostabolin-C™ and PTH 1-30, are administered subcutaneously to rats for 104 weeks at doses of 0.5, 5, 30, and 50 μg/kg/day. The test article is administered subcutaneously. Analysis of the incidence and morphology of tumours following administration may demonstrate that administration of the PTH peptides of the present invention over the course of two years may lead to lower incidences of osteosarcomas as compared to administration of a similar duration of prior art PTH peptides. This difference could be due to the different amino acids sequences and/or to the different signalling pathways activated by the PTH molecules.

### EXAMPLE 11

### Comparison of Ostabolin-C and Forteo

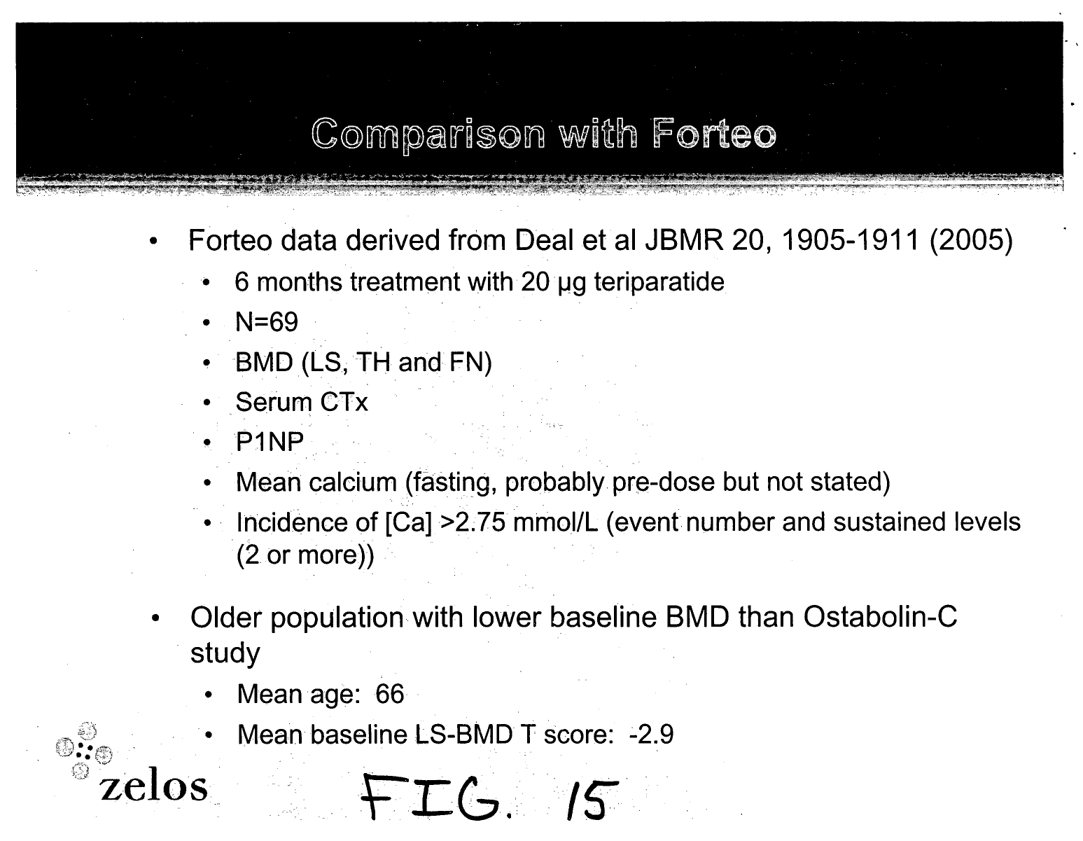

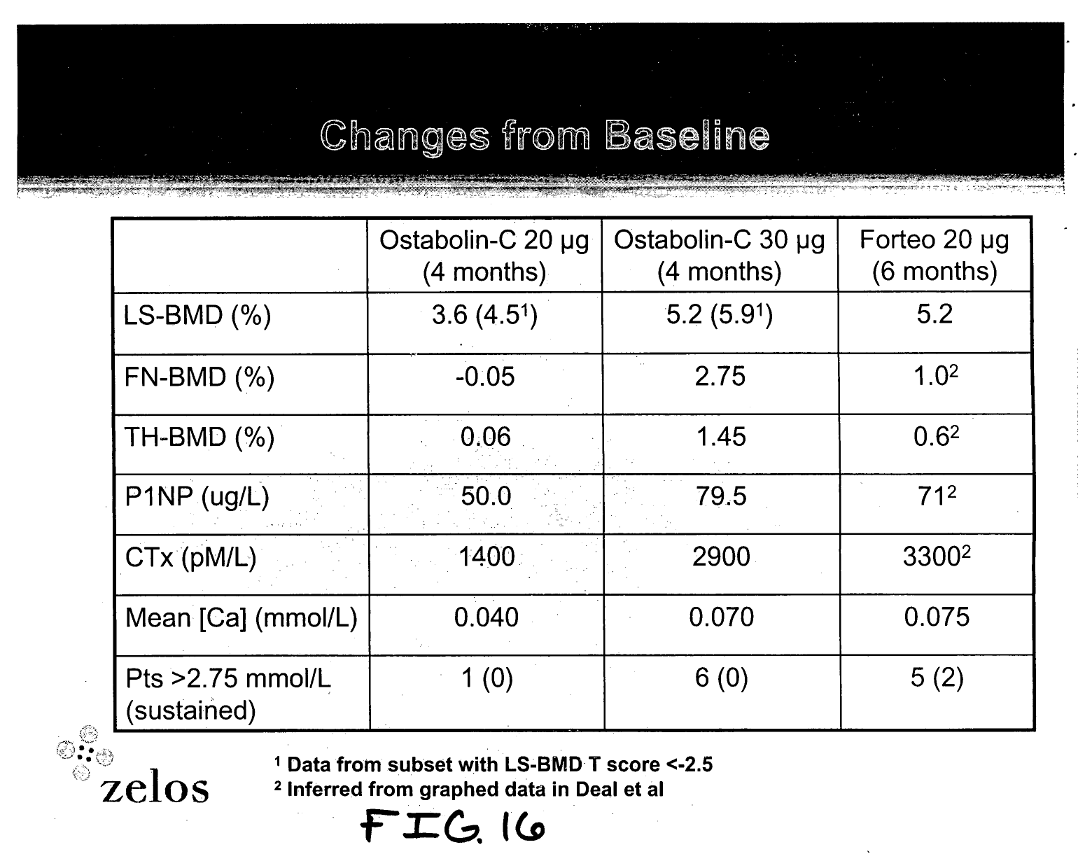

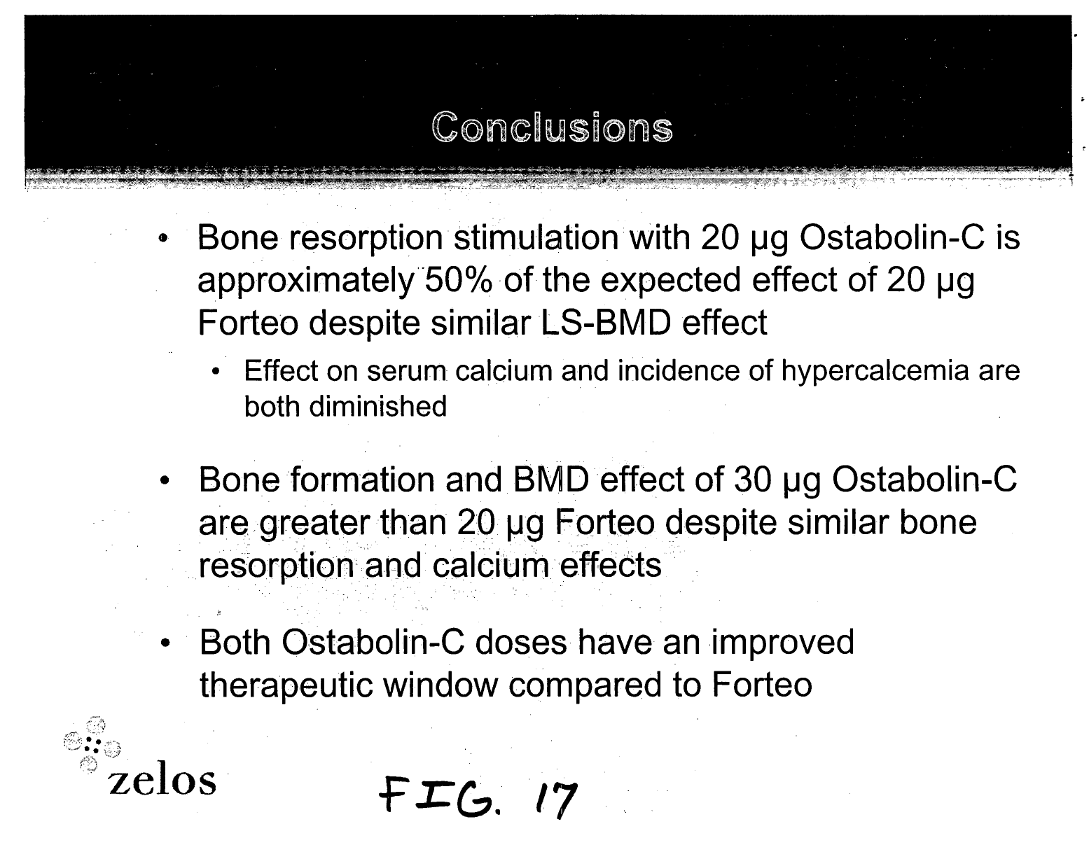

The below table illustrates a comparison of Ostabolin-C with Forteo data derived from Deal et al., (2005) J. Bone Min. Res. 20, p. 1905-1991. As shown below, bone resorption stimulation with 20 μg Ostabolin-C is approximately 50% of the expected effect of 20 μg Forteo despite similar effects on LS-BMD. The effect of 20 pg Ostabolin-C on serum calcium and incidence of hypercalcemia are both diminished. The effect of 30 μg Ostabolin-C on bone formation and BMD is greater than the effect of 20 μg Forteo despite similar effects on bone resorption and calcium. Both 20 μg and 30 μg Ostabolin-C doses have an improved therapeutic window compared to Forteo. These results are also represented in FIGS. 15, 16, and 17.

| | Ostabolin-C | Ostabolin-C | |
|---|---|---|---|
| | 20 μg | 30 μg | Forteo 20 μg |
| | (4 months) | (4 months) | (6 months) |
| LS-BMD (%) | 3.6 (4.51) | 5.2 (5.9¹) | 5.2 |
| FN-BMD (%) | −0.05 | 2.75 | 1.02 |
| TH-BMD (%) | 0.06 | 1.45 | 0.6² |
| P1NP (μg/L) | 50.0 | 79.5 | 71² |
| CTx (pM/L) | 1400 | 2900 | 3300² |
| Mean [Ca](mmol/L) | 0.040 | 0.070 | 0.075 |
| Pts > 2.75 mmol/L | 1 (0) | 6 (0) | 5 (2) |
| (sustained) | | | |

¹Data from subset with LS-BMD T score <−2.5.
²Inferred from graphed data in Deal et al. (2005) J. Bone Min. Res. 20, p. 1905-1991.

While this invention has been particularly shown and described with references to preferred embodiment thereof, it will be understood by those skilled in the art that various changes in form and details may be made therein without departing from the scope of the invention encompassed by the appended claims.

What is claimed is:

1. A method for the treatment of osteoporosis, comprising administering to a subject in need thereof a pharmaceutically acceptable formulation comprising a parathyroid hormone (PTH) peptide analogue in a daily dose of 2 μg to 60 μg, wherein said PTH peptide analogue has a reduced phospholipase-C activity and maintains adenylate cyclase activity.

2. The method of claim 1, wherein said human subject is a man or a woman.

3. The method of claim 2, wherein said woman is post-menopausal.

4. The method of claim 1, wherein said osteoporosis is selected from the group consisting of: advanced-stage osteoporosis; hypogonadal osteoporosis; spinal osteoporosis; transplant-induced osteoporosis, and steroid-induced osteoporosis.

5. The method of claim 1, wherein supplemental vitamin D is concurrently administered.

6. The method of claim 1, wherein supplemental calcium is concurrently administered.

7. The method of claim 1, wherein supplemental vitamin D and calcium are concurrently administered.

8. The method of claim 1, wherein the PTH peptide analogue is selected from the group consisting of: PTH-(1-31) peptide analogues, and PTH-(1-30) peptide analogues.

9. The method of claim 1, wherein the PTH peptide analogue is selected from the group consisting of PTH-(1-31)NH2; PTH-(1-30)NH2; PTH-(1-29)NH2; PTH-(1-28)NH2; Leu27PTH-(1-31)NH2; Leu27PTH-(1-30)NH2; Leu27PTH-(1-29)NH2; Leu27cyclo(22-26)PTH-(1-31)NH2 Ostabolin-C™; Leu27cyclo(22-26)PTH-(1-34)NH2; Leu27cyclo(Lys26-Asp30)PTH-(1-34)NH2; Cyclo(Lys27-Asp30)PTH-(1-34)NH2; Leu27cyclo(22-26)PTH-(1-31)NH2; Ala27 or Nle27 or Tyr27 or Ile27cyclo(22-26)PTH-(1-31)NH2; Leu27cyclo(22-26)PTH-(1-32)NH2; Leu27cyclo(22-26)PTH-(1-31)OH; Leu27cyclo(26-30)PTH-(1-31)NH2; Cys22Cys26Leu27cyclo(22-26)PTH-(1-31)NH2; Cys22Cys26Leu27cyclo(26-30)PTH-(1-31)NH2; Cyclo(27-30)PTH-(1-31)NH2; Leu27cyclo(22-26)PTH-(1-30)NH2; Cyclo(22-26)PTH-(1-31)NH2; Cyclo(22-26)PTH-(1-30)NH2; Leu27cyclo(22-26)PTH-(1-29)NH2; Leu27cyclo(22-26)PTH-(1-28)NH2; Glu17,Leu27cyclo(13-17)(22-26)PTH-(1-28)NH2; and Glu17,Leu27cyclo(13-17)(22-26)PTH-(1-31)NH2.

10. The method of claim 1, wherein the treatment comprises increasing bone mineral density and reducing the risk of vertebral and non-vertebral fracture.

11. The method of claim 1, wherein the daily dose of PTH peptide analogue is selected from the group consisting of: 5 μg, 10 μg, 15 μg, 20 μg, 25 μg, and 30 μg.

12. The method of claim 1, wherein the daily dose of hPTH peptide analogue administered results in a maximum plasma concentration of the peptide is selected from the group consisting of 10 to 400 pg/mL, 20 to 300 pg/mL, 50 to 280 pg/mL, 80 to 250 pg/mL, and 100 to 150 pg/mL.

13. The method of claim 1, wherein the administration is selected from the group consisting of oral, topical, pulmonary, transdermal, intranasal, transpercutaneous, parenteral injection and subcutaneous injection.

14. The method of claim 1, wherein said PTH peptide analogue induces bone formation and leads to a bone resorption level which is less than the bone resorption level following administration of PTH peptides 34 amino acid residues in length or longer.

15. The method of claim 14, wherein bone resorption is measured by the level of bone resorption markers.

16. The method of claim 15, wherein the bone resorption markers are selected from the group consisting of C-terminal telopeptide (CTx), N-telopeptide (NTx). pyridinoline, deoxypyridinoline, and urinary deoxypyridinoline (urinary DPD).

17. The method of claim 14, wherein the bone formation is measured by the level of bone formation markers.

18. The method of claim 17, wherein the bone formation markers comprise osteocalcin, amino terminal pro-peptide of type I pro-collagen (P1NP), and bone-specific alkaline phosphatase (BSAP).

19. The method of claim 1, wherein bone formation is increased and any increase in serum calcium levels are less than from 1% to 25% change from baseline.

20. A method for treating a bone fracture, comprising administering to a subject in need thereof a pharmaceutically acceptable formulation comprising a parathyroid hormone (PTH) peptide analogue in a daily dose of 2 μg to 60 μg, wherein said PTH peptide analogue has reduced phospholipase-C activity and maintains adenylate cyclase activity.

21. The method of claim 20, wherein said fracture occurs at a site selected from the group consisting of: the hip, forearm, humerus, wrist, radius, tibia, femur, ankle, rib, and foot.

22. The method of claim 20, wherein the subject has osteoporosis or other bone degenerative disease.

23. The method of claim 20, wherein the PTH peptide analogue is selected from the group consisting of: PTH-(1-31) peptide analogues and PTH-(1-30) peptide analogues.

24. The method of claim 20, wherein the PTH peptide analogue is selected from the group consisting of PTH-(1-31)NH2; PTH-(1-30)NH2; PTH-(1-29)NH2; PTH-(1-28)NH2; Leu27PTH-(1-31)NH2; Leu27PTH-(1-30)NH2; Leu27PTH-(1-29)NH2; Leu27cyclo(22-26)PTH-(1-31)NH2 Ostabolin-C™; Leu27cyclo(22-26)PTH-(1-34)NH2; Leu27cyclo(Lys26-Asp30)PTH-(1-34)NH2; Cyclo(Lys27-Asp30)PTH-(1-34)NH2; Leu27cyclo(22-26)PTH-(1-31)NH2; Ala27 or Nle27 or Tyr27 or Ile27 cyclo(22-26)PTH-(1- 31)NH2; Leu27cyclo(22-26)PTH-(1-32)NH2; Leu27cyclo(22-26)PTH-(1-31)OH; Leu27cyclo(26-30)PTH-(1-31)NH2; Cys22Cys26Leu27cyclo(22-26)PTH-(1-31)NH2; Cys22Cys26Leu27cyclo(26-30)PTH-(1-31)NH2; Cyclo(27-30)PTH-(1-31)NH2; Leu27cyclo(22-26)PTH-(1-30)NH2; Cyclo(22-26)PTH-(1-31)NH2; Cyclo(22-26)PTH-(1-30)NH2; Leu27cyclo(22-26)PTH-(1-29)NH2; Leu27cyclo(22-26)PTH-(1-28)NH2; Glu17,Leu27cyclo(13-17)(22-26)PTH-(1-28)NH2; and Glu17,Leu27cyclo(13-17)(22-26)PTH-(1-31)NH2.

25. The method of claim 20, wherein the bone fracture is either vertebral or non-vertebral fracture.

26. The method of claim 20, wherein the daily dose of PTH peptide analogue is selected from the group consisting of: 5 μg, 10 μg, 15 μg, 20 μg, 25 μg, and 30 μg.

27. The method of claim 20, wherein the daily dose of hPTH peptide analogue administered results in a maximum plasma concentration of the peptide is selected from the group consisting of 10 to 400 pg/mL, 20 to 300 pg/mL, 50 to 280 pg/mL, 80 to 250 pg/mL, and 100 to 150 pg/mL.

28. The method of claim 20, wherein the administration is oral, topical, pulmonary, transdermal, intranasal, transpercutaneous, parenteral injection or subcutaneous injection.

29. The method of claim 20, wherein said PTH peptide analogue induces bone formation and leads to a bone resorption level which is less than the bone resorption level following administration of PTH peptides 34 amino acid residues in length or longer.

30. The method of claim 29, wherein bone resorption is measured by the level of bone resorption markers.

31. The method of claim 30, wherein the bone resorption markers comprise C-terminal telopeptide (CTx) and N-telopeptide (NTx), pyridinoline, deoxypyridinoline, and urinary deoxypyridinoline (urinary DPD).

32. The method of claim 29, wherein the bone formation is measured by the level of bone formation markers.

33. The method of claim 32, wherein the bone formation markers comprise osteocalcin, amino terminal pro-peptide of type I pro-collagen (P1NP), and bone-specific alkaline phosphatase (BSAP).

34. The method of claim 20, wherein bone formation is increased and any increase in serum calcium levels are less than from 1% to 25% change from baseline.

35. A method of inducing bone formation in trabecular and cortical bones, comprising administering to a subject in need thereof a pharmaceutically acceptable formulation comprising a parathyroid hormone (PTH) peptide analogue in a daily dose of 2 μg to 60 μg, wherein said PTH peptide analogue has reduced phospholipase-C activity and maintains adenylate cyclase activity.

36. The method of claim 35, wherein said bone formation occurs at a site selected from the group consisting of: the spine, skull, ribs, hip, tibia, fibia, femur, humerus, ankle, and wrist.

37. The method of claim 35, wherein the subject has osteoporosis or other bone degenerative disease.

38. The method of claim 35, wherein the PTH peptide analogue is selected from the group consisting of: PTH-(1-31) peptide analogues and PTH-(1-30) peptide analogues.

39. The method of claim 35, wherein the PTH peptide analogue is selected from the group consisting of PTH-(1-31)NH2; PTH-(1-30)NH2; PTH-(1-29)NH2; PTH-(1-28)NH2; Leu27PTH-(1-31)NH2; Leu27PTH-(1-30)NH2; Leu27PTH-(1-29)NH2; Leu27cyclo(22-26)PTH-(1-31)NH2 Ostabolin-C™; Leu27cyclo(22-26)PTH-(1-34)NH2; Leu27cyclo(Lys26-Asp30)PTH-(1-34)NH2; Cyclo(Lys27-Asp30)PTH-(1-34)NH2; Leu27cyclo(22-26)PTH-(1-31)NH2; Ala27 or Nle27 or Tyr27 or Ile27 cyclo(22-26)PTH-(1-31)NH2; Leu27cyclo(22-26)PTH-(1-32)NH2; Leu27cyclo(22-26)PTH-(1-31)OH; Leu27cyclo(26-30)PTH-(1-31)NH2; Cys22Cys26Leu27cyclo(22-26)PTH-(1-31)NH2; Cys22Cys26Leu27cyclo(26-30)PTH-(1-31)NH2; Cyclo(27-30)PTH-(1-31)NH2; Leu27cyclo(22-26)PTH-(1-30)NH2; Cyclo(22-26)PTH-(1-31)NH2; Cyclo(22-26)PTH-(1-30)NH2; Leu27cyclo(22-26)PTH-(1-29)NH2; Leu27cyclo(22-26)PTH-(1-28)NH2; Glu17, Leu27cyclo(13-17)(22-26)PTH-(1-28)NH2; and Glu17, Leu27cyclo(13-17)(22-26)PTH-(1-31)NH2.

40. The method of claim 35, wherein the bone formation is in vertebral or non-vertebral bone.

41. The method of claim 35, wherein the daily dose of PTH peptide analogue is selected from the group consisting of: 5 μg, 10 μg, 15 μg, 20 μg, 25 μg, and 30 μg.

42. The method of claim 41, wherein the daily dose of PTH peptide analogue is 5 μg and wherein any increase in serum calcium levels of said patient is less than 10% change from baseline levels.

43. The method of claim 41, wherein the daily dose of PTH peptide analogue is 10 μg and wherein any increase in serum calcium levels of said patient is less than 5% change from baseline levels.

44. The method of claim 41, wherein the daily dose of PTH peptide analogue is 20 μg and wherein any increase in serum calcium levels of said patient is less than 3% change from baseline levels.

45. The method of claim 41, wherein the daily dose of PTH peptide analogue is 30 μg and wherein any increase in serum calcium levels of said patient is less than 25% change from baseline levels.

46. The method of claim 35, wherein the daily dose of PTH peptide analogue is 2-20 μg and the level of cortical bone formation in the subject increases by 50% to 150% from baseline.

47. The method of claim 35, wherein the daily dose of PTH peptide analogue is 20-40 μg and the level of trabecular bone formation in the subject increases by 50% to 150% from baseline.

48. The method of claim 35, wherein the daily dose of PTH peptide analogue administered results in a maximum plasma concentration of the peptide is selected from the group consisting of 10 to 400 pg/mL, 20 to 300 pg/mL, 50 to 280 pg/mL, 80 to 250 pg/mL, and 100 to 150 pg/mL.

49. The method of claim 35, wherein the administration is oral, topical, pulmonary, transdermal, intranasal, transpercutaneous, parenteral injection or subcutaneous injection.

50. The method of claim 35, wherein said PTH peptide analogue induces bone formation and leads to a bone resorption level which is less than the bone resorption level following administration of PTH peptides 34 amino acid residues in length or longer.

51. The method of claim 50, wherein bone resorption is measured by the level of bone resorption markers.

52. The method of claim 51, wherein the bone resorption markers comprise C-terminal telopeptide (CTx) and N-telopeptide (NTx), pyridinoline, deoxypyridinoline, and urinary deoxypyridinoline (urinary DPD).

53. The method of claim 35, wherein the bone formation is measured by the level of bone formation markers.

54. The method of claim 53, wherein the bone formation markers comprise osteocalcin, amino terminal pro-peptide of type I pro-collagen (P1NP), and bone-specific alkaline phosphatase (BSAP).

55. A method of treating or preventing renal osteodystrophy (ROD) and related disorders, comprising administering to a subject in need thereof a pharmaceutically acceptable formulation comprising a parathyroid hormone (PTH) peptide analogue in a daily dose of 2 μg to 60 μg, wherein said PTH peptide analogue has reduced phospholipase-C activity and maintains adenylate cyclase activity.

56. The method of claim 55, wherein said related disorders are osteitis fibrosa cystica and adynamic bone disease.

57. The method of claim 55, wherein the PTH peptide analogue is selected from the group consisting of: PTH-(1-31) peptide analogues and PTH-(1-30) peptide analogues.

58. The method of claim 55, wherein the PTH peptide analogue is selected from the group consisting of PTH-(1-31)NH2; PTH-(1-30)NH2; PTH-(1-29)NH2; PTH-(1-28)NH2; Leu27PTH-(1-31)NH2; Leu27PTH-(1-30)NH2; Leu27PTH-(1-29)NH2; Leu27cyclo(22-26)PTH-(1-31)NH2 Ostabolin-C™; Leu27cyclo(22-26)PTH-(1-34)NH2; Leu27cyclo(Lys26-Asp30)PTH-(1-34)NH2; Cyclo(Lys27-Asp30)PTH-(1-34)NH2; Leu27cyclo(22-26)PTH-(1-31)NH2; Ala27 or Nle27 or Tyr27 or Ile27 cyclo(22-26)PTH-(1-31)NH2; Leu27cyclo(22-26)PTH-(1-32)NH2; Leu27cyclo(22-26)PTH-(1-31)OH; Leu27cyclo(26-30)PTH-(1-31)NH2; Cys22Cys26Leu27cyclo(22-26)PTH-(1-31)NH2; Cys22Cys26Leu27cyclo(26-30)PTH-(1-31)NH2; Cyclo(27-30)PTH-(1-31)NH2; Leu27cyclo(22-26)PTH-(1-30)NH2; Cyclo(22-26)PTH-(1-31)NH2; Cyclo(22-26)PTH-(1-30)NH2; Leu27cyclo(22-26)PTH-(1-29)NH2; Leu27cyclo(22-26)PTH-(1-28)NH2; Glu17, Leu27cyclo(13-17)(22-26)PTH-(1-28)NH2; and Glu17,Leu27cyclo(13-17)(22-26)PTH-(1-31)NH2.

59. The method of claim 55, wherein the daily dose of PTH peptide analogue is selected from the group consisting of: 5 μg, 10 μg, 15 μg, 20 μg, 25 μg, and 30 μg.

60. The method of claim 55, wherein the daily dose of PTH peptide analogue administered results in a maximum plasma concentration of the peptide in the range of 10 to 400 pg/mL.

61. The method of claim 55, wherein the administration is oral, topical, transdermal, intranasal, transpercutaneous, parenteral injection or subcutaneous injection.

62. The method of claim 55, wherein said PTH peptide analogue induces bone formation and leads to a bone resorption level which is less than the bone resorption level following administration of PTH peptides 34 amino acid residues in length or longer.

63. The method of claim 62, wherein bone resorption is measured by the level of bone resorption markers.

64. The method of claim 63, wherein the bone resorption markers comprise C-terminal telopeptide (CTx), N-telopeptide (NTx), pyridinoline, deoxypyridinoline, and urinary deoxypyridinoline (urinary DPD).

65. The method of claim 62, wherein the bone formation is measured by the level of bone formation markers.

66. The method of claim 65, wherein the bone formation markers comprise osteocalcin, amino terminal pro-peptide of type I pro-collagen (P1NP), and bone-specific alkaline phosphatase (BSAP).

67. The method of claim 55, wherein bone formation is increased and any increase in serum calcium levels are less than from 1% to 25% change from baseline.

68. The method of claim 67 wherein the increase in bone formation is in either vertebral or non-vertebral bone.

69. A pharmaceutical formulation comprising:
a) a unit dosage form of a therapeutically effective amount of a parathyroid hormone (PTH) peptide analogue in a dosage range of 2 to 60 μg, wherein said PTH peptide analogue has reduced phospholipase-C activity and maintains adenylate cyclase activity; and
b) a pharmaceutically acceptable excipient, diluent, or carrier, or combinations thereof.

70. The pharmaceutical formulation of claim 69, wherein the PTH peptide analogue is selected from the group consisting of: PTH-(1-31) peptide analogues and PTH-(1-30) peptide analogues.

71. The pharmaceutical formulation of claim 70, wherein the PTH peptide analogue is selected from the group consisting of PTH-(1-31)NH2; PTH-(1-30)NH2; PTH-(1-29)NH2; PTH-(1-28)NH2; Leu27PTH-(1-31)NH2; Leu27PTH-(1-30)NH2; Leu27PTH-(1-29)NH2; Leu27cyclo(22-26)PTH-(1-31)NH2 Ostabolin-C™; Leu27cyclo(22-26)PTH-(1-34)NH2; Leu27cyclo(Lys26-Asp30)PTH-(1-34)NH2; Cyclo(Lys27-Asp30)PTH-(1-34)NH2; Leu27cyclo(22-26)PTH-(1-31)NH2; Ala27 or Nle27 or Tyr27 or Ile27 cyclo(22-26)PTH-(1-31)NH2; Leu27cyclo(22-26)PTH-(1-32)NH2; Leu27cyclo(22-26)PTH-(1-31)OH; Leu27cyclo(26-30)PTH-(1-31)NH2; Cys22Cys26Leu27cyclo(22-26)PTH-(1-31)NH2; Cys22Cys26Leu27cyclo(26-30)PTH-(1-31)NH2; Cyclo(27-30)PTH-(1-31)NH2; Leu27cyclo(22-26)PTH-(1-30)NH2; Cyclo(22-26)PTH-(1-31)NH2; Cyclo(22-26)PTH-(1-30)NH2; Leu27cyclo(22-26)PTH-(1-29)NH2; Leu27cyclo(22-26)PTH-(1-28)NH2; Glu17, Leu27cyclo(13-17)(22-26)PTH-(1-28)NH2; and Glu17, Leu27cyclo(13-17)(22-26)PTH-(1-31)NH2.

72. The pharmaceutical formulation of claim 69, wherein the unit dosage is selected from the group consisting of: 5 μg; 10 μg; 15 μg, 20 μg; 25 μg, and 30 μg.

73. The pharmaceutical formulation of claim 69, wherein the therapeutically effective amount of the PTH peptide analogue results in a maximum plasma concentration of the peptide is selected from the group consisting of 10 to 400 pg/mL, 20 to 300 pg/mL, 50 to 280 pg/mL, 80 to 250 pg/mL, and 100 to 150 pg/mL.

74. The pharmaceutical formulation of claim 69 further comprising one or more bone enhancing agents.

75. The pharmaceutical formulation of claim 74, wherein the one or more bone enhancing agents is selected from the group consisting of: selective estrogen receptor modulators (SERMs) natural or synthetic hormones; growth factors; vitamins; minerals; isoflavones; statin drugs; agonsists or antagonsists of receptors on the surface of osteoblasts and osteoclasts; bisphosphonate; and anabolic bone agents.

76. A kit for treating a bone deficit disorder comprising, in one or more containers, a therapeutically effective amount of the pharmaceutical composition of claim 85 contained in a device, and a label or packaging insert containing instructions for use.

77. The kit of claim 76, wherein the pharmaceutical composition is provided as a liquid and wherein the device comprises one or more pre-filled syringes.

78. The kit of claim 76, wherein the device comprises a disposable cartridge assembly for use with a medication delivery pen.

79. The kit of claim 78, wherein the pharmaceutical composition is provided as a liquid or in a lyophilized form that is reconstituted prior to use.

80. The kit of claim 78, wherein the cartridge assembly has the capacity to hold from 1 to 60 daily doses.

81. A method of administering the pharmaceutical formulation of claim 69 to a subject for treating osteoporosis, for treating or preventing a bone fracture, for inducing bone formation in trabecular and cortical bones, for treating or preventing renal osteodystrophy (ROD) and related disorders, or for any other therapeutic use of PTH wherein calcium monitoring of the subject post-administration is not required.

82. A method of administering the pharmaceutical formulation of claim 69 to a subject for treating osteoporosis, for treating or preventing a bone fracture, for inducing bone formation in trabecular and cortical bones, for treating or preventing renal osteodystrophy (ROD) and related disorders, or for any other therapeutic use of PTH, wherein a warning to the subject regarding possible osteosarcoma formation in the subject is not required.
